(19) United States
(12) Patent Application Publication
(10) Pub. No.: US 2011/0028489 A1
(43) Pub. Date: Feb. 3, 2011

(54) Pyrimidine Derivatives and Their Use for Treating Bone-Related Disorders

(76) Inventors: Lars Andersson, Sodertalje (SE); Stefan Berg, Sodertalje (SE); Jeremy Burrows, Sodertalje (SE); Sven Hellberg, Sodertalje (SE); Fernando Huerta, Sodertalje (SE); Tobias Rein, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(21) Appl. No.: 12/856,879

(22) Filed: Aug. 16, 2010

Related U.S. Application Data
(63) Continuation of application No. 12/088,899, filed on Apr. 1, 2008.

(30) Foreign Application Priority Data
Oct. 3, 2005 (SE) 0502177-9

(51) Int. Cl. A61K 31/497; C07D 233/22; C07D 401/14; C07D 241/04; C07D 403/14; A61K 31/4164; A61K 31/506; A61K 31/4965; A61P 19/00

(52) U.S. Cl. 514/252.19; 548/333.5; 544/331; 544/383; 544/295; 514/399; 514/275; 514/255.02

(57) ABSTRACT

The present invention relates to a new use of pyrimidine derivatives of formula I, as a free base or a pharmaceutically acceptable salt thereof in the manufacture of a medicament in the treatment and/or prophylaxis of Alzheimer's Disease:

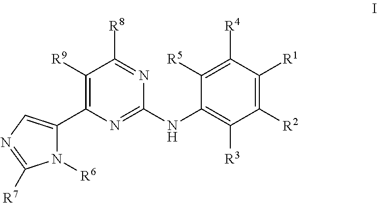

### TECHNICAL FIELD OF INVENTION

The present invention relates to a new use of pyrimidine derivatives, as a free base or a pharmaceutically acceptable salt thereof in the manufacture of a medicament in the treatment and/or prophylaxis of conditions associated with glycogen synthase kinase-3. The present invention further relates to a method of treatment and/or prophylaxis of conditions associated with glycogen synthase kinase-3, comprising administering to a mammal, including man in need of such prevention and/or prophylaxis a therapeutically effective amount of said pyrimidine derivatives. In addition, the present invention relates to new compounds suitable for the inhibition of glycogen synthase kinase-3.

### BACKGROUND OF THE INVENTION

Glycogen synthase kinase 3 (GSK3) is a serine/threonine protein kinase composed of two isoforms (α and β), which are encoded by distinct genes but are highly homologous within the catalytic domain. GSK3 is highly expressed in the central and peripheral nervous system. GSK3 phosphorylates several substrates including tau, β-catenin, glycogen synthase, pyruvate dehydrogenase and elongation initiation factor 2b (eIF2b). Insulin and growth factors activate protein kinase B, which phosphorylates GSK3 on serine 9 residue and inactivates it.

### Alzheimer's Disease (AD) Dementias, and Taupathies

AD is characterized by cognitive decline, cholinergic dysfunction and neuronal death, neurofibrillary tangles and senile plaques consisting of amyloid-β deposits. The sequence of these events in AD is unclear, but they are believed to be related. Glycogen synthase kinase 3β (GSK3β) or Tau (τ) phosphorylating kinase selectively phosphorylates the microtubule associated protein τ in neurons at sites that are hyperphosphorylated in AD brains. Hyperphosphorylated protein τ has lower affinity for microtubules and accumulates as paired helical filaments, which are the main components that constitute neurofibrillary tangles and neuropil threads in AD brains. This results in depolymerization of microtubules, which leads to dying back of axons and neuritic dystrophy. Neurofibrillary tangles are consistently found in diseases such as AD, amyotrophic lateral sclerosis, parkinsonism-dementia of Gaum, corticobasal degeneration, dementia pugilistica and head trauma, Down's syndrome, postencephalatic parkinsonism, progressive supranuclear palsy, Niemann-Pick's Disease and Pick's Disease. Addition of amyloid-β to primary hippocampal cultures results in hyperphosphorylation of τ and a paired helical filaments-like state via induction of GSK3β activity, followed by disruption of axonal transport and neuronal death (Imahori and Uchida., J. Biochem 121:179-188, 1997). GSK3β preferentially labels neurofibrillary tangles and has been shown to be active in pre-tangle neurons in AD brains. GSK3 protein levels are also increased by 50% in brain tissue from AD patients. Furthermore, GSK3β phosphorylates pyruvate dehydrogenase, a key enzyme in the glycolytic pathway and prevents the conversion of pyruvate to acetyl-Co-A (Hoshi et al., PNAS 93:2719-2723, 1996). Acetyl-Co-A is critical for the synthesis of acetylcholine, a neurotransmitter with cognitive functions. Thus, GSK3β inhibition may have beneficial effects in progression as well as the cognitive deficits associated with Alzheimer's disease and other above-referred to diseases.

### Chronic and Acute Neurodegenerative Diseases

Growth factor mediated activation of the PI3K/Akt pathway has been shown to play a key role in neuronal survival. The activation of this pathway results in GSK3β inhibition. Recent studies (Bhat et. al., PNAS 97:11074-11079 (2000)) indicate that GSK3β activity is increased in cellular and animal models of neurodegeneration such as cerebral ischemia or after growth factor deprivation. For example, the active site phosphorylation was increased in neurons vulnerable to apoptosis, a type of cell death commonly thought to occur in chronic and acute degenerative diseases such as Alzheimer's Disease, Parkinson's Disease, amyotrophic lateral sclerosis, Huntington's Disease and HIV dementia, ischemic stroke and head trauma. Lithium was neuroprotective in inhibiting apoptosis in cells and in the brain at doses that resulted in the inhibition of GSK3β. Thus GSK3β inhibitors could be useful in attenuating the course of neurodegenerative diseases.

### Bipolar Disorders (BD)

Bipolar Disorders are characterised by manic episodes and depressive episodes. Lithium has been used to treat BD based on its mood stabilising effects. The disadvantage of lithium is the narrow therapeutic window and the danger of overdosing that can lead to lithium intoxication. The recent discovery that lithium inhibits GSK3 at therapeutic concentrations has raised the possibility that this enzyme represents a key target of lithium's action in the brain (Stambolic et al., Curr. Biol. 6:1664-1668, 1996; Klein and Melton; PNAS 93:8455-8459, 1996). Inhibition of GSK3β may therefore be of therapeutic relevance in the treatment of BD as well as in AD patients that have affective disorders.

### Schizophrenia

GSK3 is involved in signal transduction cascades of multiple cellular processes, particularly during neural development. Kozlovsky et al (Am J Psychiatry 2000 May; 157(5):831-3) found that GSK3β levels were 41% lower in the schizophrenic patients is than in comparison subjects. This study indicates that schizophrenia involves neurodevelopmental pathology and that abnormal GSK3 regulation could play a role in schizophrenia. Furthermore, reduced β-catenin levels have been reported in patients exhibiting schizophrenia (Cotter et al., Neuroreport 9:1379-1383 (1998)).

### Diabetes

Insulin stimulates glycogen synthesis in skeletal muscles via the dephosphorylation and thus activation of glycogen synthase. Under resting conditions, GSK3 phosphorylates and inactivates glycogen synthase via dephosphorylation. GSK3 is also over-expressed in muscles from Type II diabetic patients (Nikoulina et al., Diabetes 2000 February; 49(2):263-71). Inhibition of GSK3 increases the activity of glycogen synthase thereby decreasing glucose levels by its conversion to glycogen. GSK3 inhibition may therefore be of therapeutic relevance in the treatment of Type I and Type II diabetes and diabetic neuropathy.

### Hair Loss

GSK3 phosphorylates and degrades β-catenin. β-catenin is an effector of the pathway for keratonin synthesis. β-catenin stabilisation may be lead to increase hair development. Mice expressing a stabilised β-catenin by mutation of sites phosphorylated by GSK3 undergo a process resembling de novo hair morphogenesis (Gat et al., Cell 1998 Nov. 25; 95 (5):605-14)). The new follicles formed sebaceous glands and dermal papilla, normally established only in embryogenesis. Thus GSK3 inhibition may offer treatment for baldness.

### Oral Contraceptives

Vijajaraghavan et al. (Biol Reprod 2000 June; 62 (6):1647-54) reported that GSK3 is high in motile versus immotile sperm. Immunocytochemistry revealed that GSK3 is present in the flagellum and the anterior portion of the sperm head. These data suggest that GSK3 could be a key element underlying motility initiation in the epididymis and regulation of mature sperm function. Inhibitors of GSK3 could be useful as contraceptives for males.

### Bone-Related Disorders

It has been shown that GSK3 inhibitors could be used for treatment of bone-related disorders. This has been discussed in e.g. Tobias et al., Expert Opinion on Therapeutic Targets, February 2002, pp 41-56.

### DISCLOSURE OF THE INVENTION

The object of the present invention is to provide the use of compounds having a selective inhibiting effect at GSK3 as well as having a good bioavailability.

The present invention relates to the use of a compound of formula I,

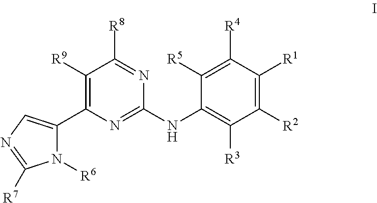

wherein
R¹ is selected from hydrogen, halo, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};
R² and R⁴ are independently selected from hydrogen, halo, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};
R³ and R⁵ independently are selected from hydrogen, C₁₋₃alkyl, C₁₋₃haloalkyl and OR^{a};
R⁶ is selected from C₂₋₄alkyl, C₂₋₄alkenyl, C₂₋₄alkynyl, and C₂₋₄haloalkyl;
R⁷ is selected from C₁₋₃alkyl, CN, and C₁₋₃haloalkyl, said C₁₋₃alkyl or C₁₋₃haloalkyl optionally substituted with one or more OR^{a};
R⁸ and R⁹ are independently selected from hydrogen, CN and halo;
R^{a} is hydrogen, C₁₋₃alkyl or C₁₋₃haloalkyl, said C₁₋₃alkyl or C₁₋₃haloalkyl optionally substituted with one or more C₁₋₃alkoxy;
R^{b} and R^{c} are independently selected from hydrogen, C₁₋₆alkyl or C₁₋₆haloalkyl, said C₁₋₆alkyl or C₁₋₆haloalkyl optionally substituted with one or more OR^{a} or NR^{d}R^{e}; or
R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;
R^{d} and R^{e} are independently selected from hydrogen, C₁₋₆alkyl or C₁₋₆haloalkyl, said
C₁₋₆alkyl or C₁₋₆haloalkyl optionally substituted with one or more OR^{a}; or
R^{d} and R^{e} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;
R^{h} is hydrogen, C₁₋₃alkyl or C₁₋₃haloalkyl, said C₁₋₃alkyl or C₁₋₃haloalkyl optionally substituted with one or more C₁₋₃alkoxy;
Rⁱ is C₁₋₃alkyl or C₁₋₃haloalkyl, said C₁₋₃alkyl or C₁₋₃haloalkyl optionally substituted with one or more OR^{a}; and
R^{j} is an aryl or heteroaryl ring, wherein said aryl or heteroaryl ring is optionally substituted with one or more C₁₋₃alkyl, OR^{a}, halo or CN;
or a pharmaceutically acceptable salt thereof for use in the manufacturing of a medicament for prevention and/or treatment of Alzheimer's Disease.

One embodiment of the present invention relates to the use of a compound according to formula I, wherein

R¹ is selected from hydrogen, SO₂NR^{b}R^{c}, C(O)NR^{b}R^{c}, CH₂NR^{b}R^{c} and C(O)R^{j};
is R² and R⁴ are independently selected from hydrogen, halo, CN, C₁₋₃alkyl, OR^{a}, and SO₂Rⁱ;
R³ and R⁵ independently are selected from hydrogen, C₁₋₃alkyl, C₁₋₃haloalkyl;
R⁶ is selected from C₂₋₄alkyl, C₂₋₄alkenyl, C₂₋₄alkynyl, and C₂₋₄haloalkyl;
R⁷ is C₁₋₃alkyl;
R⁸ and R⁹ are independently selected from hydrogen and halo; and
R^{a} is C₁₋₃alkyl or C₁₋₃haloalkyl, said C₁₋₃alkyl or C₁₋₃haloalkyl optionally substituted with one or more C₁₋₃alkoxy;
R^{b} and R^{c} are independently selected from hydrogen, C₁₋₆alkyl or C₁₋₆haloalkyl, said C₁₋₆alkyl or C₁₋₆haloalkyl optionally substituted with one or more OR^{a}; or
R^{b} and R^{e} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;
Rⁱ is C₁₋₃alkyl; and
R^{j} is aryl or heteroaryl;
or a pharmaceutically acceptable salt thereof.

Another embodiment of the present invention relates to the use of a compound according to formula I, wherein

R¹ is selected from SO₂NR^{b}R^{e}, C(O)NR^{b}R^{e} and C(O)R^{j};
R² and R⁴ are independently selected from hydrogen, halo, CN, C₁₋₃alkyl, OR^{a}, and SO₂Rⁱ;
R³ and R⁵ independently are selected from hydrogen, C₁₋₃alkyl, C₁₋₃haloalkyl;
R⁶ is C₂₋₄alkyl;
R⁷ is C₁₋₃alkyl;
R⁸ and R⁹ are independently selected from hydrogen and halo;
R^{a} is C₁₋₃alkyl or C₁₋₃haloalkyl;
R^{b} and R^{c} may, together with the atom to which they are attached, form a 4-, 5 or 6-membered heterocyclic ring containing one or more heteroatoms selected from N, O or S, wherein said heterocyclic ring is optionally substituted with one or more C₁₋₃alkyl;
Rⁱ is C₁₋₃alkyl; and
R^{j} is aryl or heteroaryl;
or a pharmaceutically acceptable salt thereof.

Yet another embodiment of the present invention relates to the use of a compound according to formula I, wherein R⁹ is halo and R⁸ is hydrogen.

A further embodiment of the present invention relates to the use according to claim 4, wherein R⁹ is fluoro.

Another embodiment of the present invention relates to the use of a compound according to formula I, wherein R⁶ is C₂₋₄alkyl. According to one embodiment of the present invention, R⁶ is isopropyl.

One embodiment of the present invention relates to the use of a compound according to formula I, wherein R⁷ is fluoromethyl or methyl.

Another embodiment of the present invention provides the use of a compound according to formula I, wherein R² and R⁴ are hydrogen.

Yet another embodiment of the present invention relates to the use of a compound according to formula I, wherein R⁵ and R³ are hydrogen.

One embodiment of the present invention provides the use of a compound according to formula I, wherein R¹ is selected from C(O)NR^{b}R^{c}, SO₂R^{b}R^{c}, SO₂Rⁱ or C(O)R^{j}. According to one embodiment of the present invention, R^{j} is phenyl or piperidin. According to another embodiment of the present invention, R^{b} and R^{e}, together with the atom to which they are attached, form a 6-membered heterocyclic ring containing one or more heteroatoms selected from N, wherein said heterocyclic ring is optionally substituted with one or more halo, C₁₋₃alkyl or C₁₋₃haloalkyl. According to a further embodiment of the present invention, said heterocyclic ring is substituted with one or more C₁₋₃alkyl. According to yet a further embodiment if the present invention, said C₁₋₃alkyl is methyl. According to another embodiment of the present invention, Rⁱ is C₁₋₃alkyl. According to yet another embodiment of the present invention, Rⁱ is methyl,

The present invention also relates to the use of a compound of formula I, in the manufacturing of a medicament for the treatment and/or prophylaxis of conditions associated with glycogen synthase kinase-3 inhibition, said compound being selected from:
(4-{[5-Fluoro-4-(1-isopropyl-2-methyl-1H-imidazol-5-yl)pyrimidin-2-yl]amino}phenyl)(phenyl)methanone hydrochloride;
(4-{[5-Fluoro-4-(1-isopropyl-2-methyl-1H-imidazol-5-yl)pyrimidin-2-yl]amino}phenyl)(pyridin-2-yl)methanone hydrochloride;
(4-{[5-Fluoro-4-(1-isopropyl-2-methyl-1H-imidazol-5-yl)pyrimidin-2-yl]amino}phenyl)(pyridin-3-yl)methanone hydrochloride;
5-Fluoro-4-(1-isopropyl-2-methyl-1H-imidazol-5-yl)-N-{3-methyl-4-[(4-methylpiperazin-1-yl)sulfonyl]phenyl}pyrimidin-2-amine hydrochloride;
5-Fluoro-4-(1-isopropyl-2-methyl-1H-imidazol-5-yl)-N-{2-methyl-4-[(4-methylpiperazin-1-yl)sulfonyl]phenyl}pyrimidin-2-amine hydrochloride;
5-Fluoro-4-(1-isopropyl-2-methyl-1H-imidazol-5-yl)-N-[4-[(4-methylpiperazin-1-yl)sulfonyl]-2-(trifluoromethyl)phenyl]pyrimidin-2-amine hydrochloride;
5-Fluoro-4-(1-isopropyl-2-methyl-1H-imidazol-5-yl)-N-[4-[(4-methylpiperazin-1-yl)sulfonyl]-3-(trifluoromethoxy)phenyl]pyrimidin-2-amine hydrochloride;
5-Fluoro-4-(1-isopropyl-2-methyl-1H-imidazol-5-yl)-N-[4-[(4-methylpiperazin-1-yl)carbonyl]-3-(methylsulfonyl)phenyl]pyrimidin-2-amine hydrochloride;
5-{[5-Fluoro-4-(1-isopropyl-2-methyl-1H-imidazol-5-yl)pyrimidin-2-yl]amino}-2-[(4-methylpiperazin-1-yl)carbonyl]benzonitrile hydrochloride;
N-{3-Chloro-4-[(4-methylpiperazin-1-yl)carbonyl]phenyl}-5-fluoro-4-(1-isopropyl-2-methyl-1H-imidazol-5-yl)pyrimidin-2-amine hydrochloride;
5-Fluoro-4-(1-isopropyl-2-methyl-1H-imidazol-5-yl)-N-{3-methoxy-4-[(4-methylpiperazin-1-yl)carbonyl]phenyl}pyrimidin-2-amine hydrochloride;
(4-{[5-Fluoro-4-(1-isopropyl-2-methyl-1H-imidazol-5-yl)pyrimidin-2-yl]amino}phenyl)(pyridin-4-yl)methanone hydrochloride;
5-Fluoro-4-(1-isopropyl-2-methyl-1H-imidazol-5-yl)-N-{4-[(4-methylpiperazin-1-yl)carbonyl]phenyl}pyrimidin-2-amine hydrochloride;
5-Fluoro-4-(1-isopropyl-2-methyl-1H-imidazol-5-yl)-N-{4-[(4-methylpiperazin-1-yl)sulfonyl]phenyl}pyrimidin-2-amine hydrochloride;
5-Fluoro-4-[1-isopropyl-2-(trifluoromethyl)-1H-imidazol-5-yl]-N-[4-(methylsulfonyl)phenyl]pyrimidin-2-amine hydrochloride;
5-Fluoro-4-[1-isopropyl-2-(trifluoromethyl)-1H-imidazol-5-yl]-N-[4-(methylsulfonyl)phenyl]pyrimidin-2-amine hydrochloride;
{4-[5-Fluoro-4-(3-isopropyl-2-trifluoromethyl-3H-imidazol-4-yl)-pyrimidin-2-ylamino]-phenyl}-(4-methyl-piperazin-1-yl)-methanone hydrochloride; and
3-{[5-Fluoro-4-(1-isopropyl-2-methyl-1H-imidazol-5-yl)pyrimidin-2-yl]amino}-N-(3-methoxypropyl)benzamide hydrochloride;
   or a pharmaceutically acceptable salt thereof.

According to one aspect of the present invention, there is provided the use of a compound of formula I as a free base or a pharmaceutically acceptable salt thereof in the manufacture of a medicament for the treatment and/or prophylaxis of conditions associated with GSK3.

According to another aspect of the present invention, there is provided a method of treatment and/or prophylaxis of conditions associated with GSK3 comprising administering to a mammal, including man in need of such treatment and/or prophylaxis a therapeutically effective amount of a compound of formula I as a free base or a pharmaceutically acceptable salt thereof.

According to yet another aspect of the present invention, there is provided a pharmaceutical formulation for use in the treatment and/or prophylaxis of conditions associated with GSK3 comprising a therapeutically effective amount of a compound of formula (I) as a free base or a pharmaceutically acceptable salt thereof and conventional excipients.

It has now suprisingly been found that the group of pyrimidine derivatives as described below are well suited for inhibiting glycogen synthase kinase-3. The use of said glycogen synthase kinase-3 inhibitors are suitable in the treatment and/or prophylaxis of conditions associated with especially, dementia, Alzheimer's Disease, Parkinson's Disease, Frontotemporal dementia Parkinson's Type, Parkinson dementia complex of Guam, HIV dementia, diseases with associated neurofibrillar tangle pathologies, amyotrophic lateral sclerosis, corticobasal degeneration, dementia pugilistica, Down's syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, Type I and Type II Diabetes and Diabetic neuropathy, hair loss, contraceptive medication and bone disorder.

Listed below are definitions of various terms used in the specification and claims to describe the present invention.

In this specification the term “alkyl” includes both straight and branched chain as well as cyclicalkyl groups. The term C₁₋₃alkyl having 1 to 3 carbon atoms and may be, but is not limited to, methyl, ethyl, n-propyl, i-propyl, or cyclopropyl. The term C₂₋₄alkyl having 2 to 4 carbon atoms and may be, but is not limited to, ethyl, n-propyl, i-propyl, n-butyl, i-butyl, s-butyl, t-butyl, and cyclobutyl. The term C₁₋₆alkyl having 1 to 6 carbon atoms and may be, but is not limited to, methyl, ethyl, n-propyl, i-propyl, n-butyl, i-butyl, s-butyl, t-butyl, n-pentyl, i-pentyl, t-pentyl, neo-pentyl, n-hexyl, i-hexyl or cyclohexyl.

The term “alkenyl” refers to a straight or branched chain alkenyl group. The term C₂₋₄alkenyl having 2 to 4 carbon atoms and one double bond, and may be, but is not limited to, vinyl, allyl, propenyl, i-propenyl, butenyl, i-butenyl, and crotyl.

The term “alkynyl” refers to a straight or branched chain alkynyl group. The term C₂₋₄alkynyl having 2 to 4 carbon atoms and one triple bond, and may be, but is not limited to, ethynyl, propargyl, butynyl, and i-butynyl.

The term “C₁₋₃alkoxy” includes both straight and branched chains. The term “C₁₋₃alkoxy” having 1 to 3 carbon atoms and may be, but is not limited to, methoxy, ethoxy, n-propoxy, or i-propoxy.

The term “halogen” refers to fluorine, chlorine, bromine and iodine.

The term “haloalkyl” refers to an alkyl group, defined as above, in which one or several of the hydrogen substituents have been replaced by halogen substituents, in which the term halogen is defined as above.

The term “aryl” refers to an optionally substituted monocyclic or bicyclic hydrocarbon ring system containing at least one unsaturated aromatic ring. The “aryl” may be fused with a C₅₋₇cycloalkyl ring to form a bicyclic hydrocarbon ring system. Examples and suitable values of the term “aryl”, but not limiting, are phenyl, naphthyl, indanyl or tetralinyl.

As used herein, “heteroaryl” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, fluorenonyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl or heteroaromatic group has 1 to about 4, 1 to about 3, or 1 to 2 heteroatoms. In some embodiments, the heteroaryl or heteroaromatic group has 1 heteroatom.

The term “4-, 5- or 6-membered heterocyclic ring containing one or more heteroatoms independently selected from N, O, or S” refers to a mono- or bicyclic-heterocyclic ring which may be saturated or partly saturated and which may optionally contain a carbonyl function and which may be, but is not limited to, azetidinyl, imidazolidinyl, imidazolinyl, morpholinyl, piperazinyl, piperidinyl, piperidonyl, pyrazolidinyl, pyrazolinyl, pyrrolidinyl, pyrrolinyl, 1-methyl-1,4-diazepane, tetrahydropyranyl or thiomorpholinyl. In the case where the heterocyclic ring contains a heteroatom selected from S or N, these atoms may optionally be in an oxidised form such as SO or SO₂.

The term “hydrochloride” includes monohydrochloride, dihydrochloride, trihydrochloride and tetrahydrochloride salts.

A suitable pharmaceutically acceptable salt of the compound of the invention is, for example, an acid-addition salt, for example an inorganic or organic acid. In addition a suitable pharmaceutically acceptable salt of the compounds of the invention is an alkali metal salt, an alkaline earth metal salt or a salt with an organic base that affords a physiologically-acceptable cation.

Some compounds of formula I may have chiral centres and/or geometric isomeric centres (E- and Z-isomers), and it is to be understood that the invention encompasses all such optical, diastereoisomers and geometric isomers.

The present invention relates to the use of compounds of formula I as hereinbefore defined as well as to the salts thereof. Salts for use in pharmaceutical compositions will be pharmaceutically acceptable salts, but other salts may be useful in the production of the compounds of formula I.

It is to be understood that the present invention relates to any and all tautomeric forms of the compounds of formula I.

An object of the invention is to provide compounds of formula I for therapeutic use, especially compounds that are useful for the prevention and/or treatment of conditions associated with glycogen synthase kinase-3 (GSK3) in mammals including man. Particularly, compounds of formula I exhibiting a selective affinity for GSK-3.

### Methods of Preparation

Another aspect of the present invention provides a process for preparing a compound of formula I, or a pharmaceutically acceptable salt or an in vivo hydrolysable ester thereof, which process (wherein R¹, R², R³, R⁴, R⁵, R⁶, R⁷, R⁸, R⁹, R^{b}, R^{c}, R^{d} and R^{e} are, unless otherwise specified, as defined in formula I comprises of:

Process a) reacting an aminopyrimidine of formula (II):

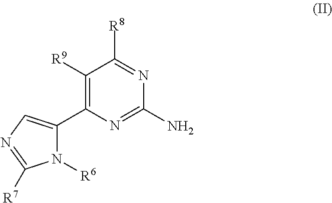

with a compound of formula (III):

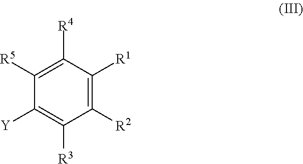

wherein Y is a displaceable group; and thereafter if necessary:
i) converting a compound of the formula I into another compound of the formula I;
ii) removing any protecting groups;
iii) forming a pharmaceutically acceptable salt or in vivo hydrolysable ester.

Y is a displaceable group, suitable values for Y are for example, a halogeno or sulphonyloxy group, for example a chloro, bromo, iodo or trifluoromethanesulphonyloxy group. According to one embodiment of the present invention Y is bromo or iodo.

Specific reaction conditions for the reaction described in Process a) are as follows. Aminopyrimidines of formula (II) and compounds of formula (III) may be reacted together under standard Buchwald-Hartwig conditions (see, for example, J. Am. Chem. Soc. 1996, 118, 7215; J. Am. Chem. Soc. 1997, 119, 8451; J. Am. Chem. Soc. 2003, 125, 6653; J. Org. Chem. 1997, 62, 1568 and 6066), for example in the presence of palladium acetate, in a suitable solvent for example an aromatic solvent such as toluene, benzene or xylene, with a suitable base for example an inorganic base such as caesium carbonate or an organic base such as potassium-t-butoxide, in the presence of a suitable ligand such as 2,2′-bis(diphenylphosphino)-1,1′-binaphthyl or 2-dicyclohexylphosphino-2′,4′,6′-triiso-propyl-1,1′-biphenyl and at a temperature in the range of +25 to +80° C.

A synthesis of aminopyrimidines of formula (II) is described in Scheme 1 (wherein R^{x} may be the same or different and is selected from C₁₋₆alkyl):

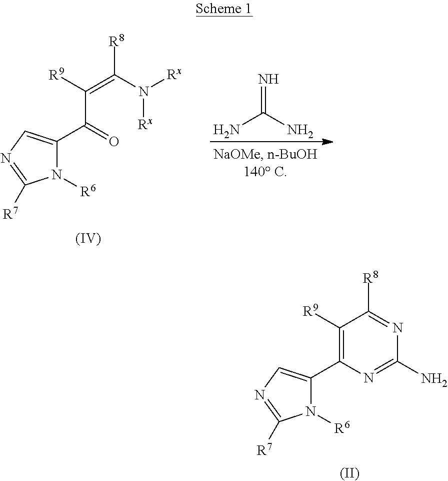

Compounds of formula (III) are commercially available compounds, or they are known in the literature, or they can be prepared by standard processes known in the art.

Compounds of formula (IV), wherein R⁶ has the general structure R^{a}—CH—R^{b}, wherein R^{a} and R^{b} are both alkyl, e.g. methyl and R⁹ is fluoro may be prepared according to Scheme 2

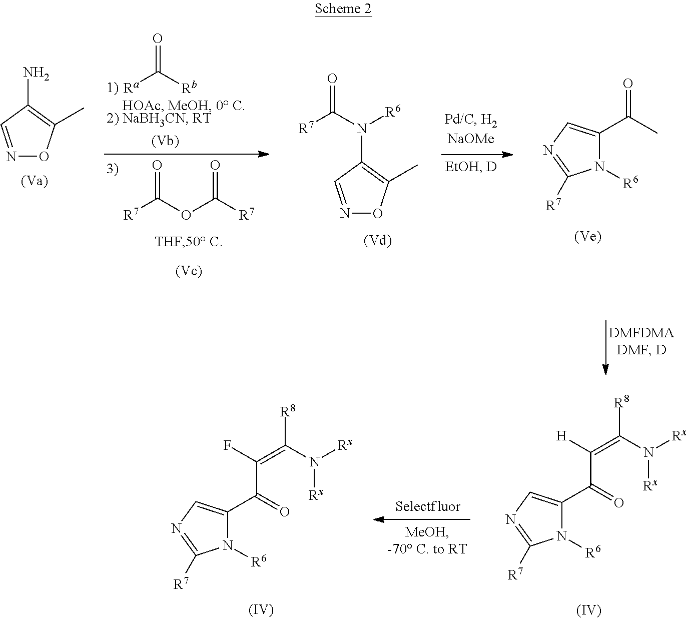

Compounds of formula (Va), (Vb) and (Vc) are commercially available compounds, or they are known in the literature, or they are prepared by standard processes known in the art.

In one aspect of the invention, there is provided a process for preparing a compound of formula I which is the process described as Process a).

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Greene, Protective Groups in Organic Synthesis, John Wiley and Sons, 1999). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

One aspect of the present invention relates to intermediates for the end products of the present invention. These intermediates are useful in the preparation of a compound of formula I as defined above. These intermediates are represented by, but not limited to, the following:
1-[4-Bromo-2-(methylsulfonyl)benzoyl]-4-methylpiperazine;
1-(4-Chloro-2-iodobenzoyl)-4-methylpiperazine;
5-Chloro-2-[(4-methylpiperazin-1-yl)carbonyl]benzonitrile;
1-(4-Chloro-2-methoxybenzoyl)-4-methylpiperazine;
2,2,2-Trifluoro-N-isopropyl-N-(5-methyl-isoxazol-4-yl)-acetamide;
5-Acetyl-2-trifluoromethyl-1-isopropyl-1H-imidazole;
(2E)-3-Dimethylamino-1-(2-trifluoromethyl-1-isopropyl-1H-imidazol-5-yl)prop-2-en-1-one;
(2Z)-3-Dimethylamino-2-fluoro-1-(2-trifluoromethyl-1-isopropyl-1H-imidazol-5-yl)prop-2-en-1-one; and
5-Fluoro-4-[2-trifluoromethyl-1-isopropyl-1H-imidazol-5-yl]pyrimidin-2-amine;
Methyl 3-{[5-fluoro-4-(1-isopropyl-2-methyl-1H-imidazol-5-yl)pyrimidin-2-yl]amino}benzoate.

### General Methods

All solvents used were analytical grade and commercially available anhydrous solvents were routinely used for reactions. Reactions were typically run under an inert atmosphere of nitrogen or argon.

¹H, ¹⁹F and ¹³C NMR spectra were recorded on a Varian Unity+ 400 NMR Spectrometer equipped with a 5 mm BBO probehead with Z-gradients, or a Varian Gemini 300 NMR spectrometer equipped with a 5 mm BBI probehead, or a Bruker Avance 400 NMR spectrometer equipped with a 60 μl dual inverse flow probehead with Z-gradients, or a Bruker DPX400 NMR spectrometer equipped with a 4-nucleus probehead equipped with Z-gradients, or a Bruker Avance 600 NMR spectrometer equipped with a 5 mm BBI probehead with Z-gradients. Unless specifically noted in the examples, spectra were recorded at 400 MHz for proton, 376 MHz for fluorine-19 and 100 MHz for carbon-13. The following reference signals were used: the middle line of DMSO-d₆ δ 2.50 (¹H), δ 39.51 (¹³C); the middle line of CD₃OD δ 3.31 (¹H) or δ 49.15 (¹³C); CDCl₃ δ 7.26 (¹H) and the middle line of CDCl₃ δ 77.16 (¹³C) (unless otherwise indicated).

Mass spectra were recorded on a Waters LCMS consisting of an Alliance 2795 (LC), Waters PDA 2996 and a ZQ single quadrupole mass spectrometer. The mass spectrometer was equipped with an electrospray ion source (ESI) operated in a positive or negative ion mode. The capillary voltage was 3 kV and cone voltage was 30 V. The mass spectrometer was scanned between m/z 100-700 with a scan time of 0.3 s. Separations were performed on either Waters X-Terra MS C8 (3.5 μm, 50 or 100 mm×2.1 mm i.d.) or an ACE 3 AQ (100 mm×2.1 mm i.d.) obtained from ScantecLab. Flow rates were regulated to 1.0 or 0.3 mL/min, respectively. The column temperature was set to 40° C. A linear gradient was applied using a neutral or acidic mobile phase system, starting at 100% A (A: 95:5 10 mM NH₄OAc:MeCN, or 95:5 8 mM HCOOH:MeCN) ending at 100% B (MeCN).

Alternatively, mass spectra were recorded on a Waters LCMS consisting of an Alliance 2690 Separations Module, Waters 2487 Dual 1 Absorbance Detector (220 and 254 nm) and a Waters ZQ single quadrupole mass spectrometer. The mass spectrometer was equipped with an electrospray ion source (ESI) operated in a positive or negative ion mode. The capillary voltage was 3 kV and cone voltage was 30 V. The mass spectrometer was scanned between m/z 97-800 with a scan time of 0.3 or 0.8 s. Separations were performed on a Chromolith Performance RP-18e (100×4.6 mm). A linear gradient was applied starting at 95% A (A: 0.1% HCOOH (aq.)) ending at 100% B (MeCN) in 5 minutes. Flow rate: 2.0 mL/min.

Microwave heating was performed in a single-mode microwave cavity producing continuous irradiation at 2450 MHz.

HPLC analyses were performed on an Agilent HP1000 system consisting of G1379A Micro Vacuum Degasser, G1312A Binary Pump, G1367A Well plate auto-sampler, G1316A Thermostatted Column Compartment and G1315B Diode Array Detector. Column: X-Terra MS, Waters, 3.0×100 mm, 3.5 μm. The column temperature was set to 40° C. and the flow rate to 1.0 ml/min. The Diode Array Detector was scanned from 210-300 nm, step and peak width were set to 2 nm and 0.05 min, respectively. A linear gradient was applied, starting at 100% A (A: 95:5 10 mM NH₄OAc:MeCN) and ending at 100% B (B: MeCN), in 4 min.

Alternatively, HPLC analyses were performed on a Gynkotek P580 HPG consisting of gradient pump with a Gynkotek UVD 170S UV-vis.-detector equipped with a Chromolith Performance RP column (C18, 100 mm×4.6 mm). The column temperature was set to +25° C. A linear gradient was applied using MeCN/0.1 trifluoroacetic acid in MilliQ water, run from 10% to 100% MeCN in 5 minutes. Flow rate: 3 ml/min.

A typical workup procedure after a reaction consisted of extraction of the product with a solvent such as ethyl acetate, washing with water followed by drying of the organic phase over MgSO₄ or Na₂SO₄, filtration and concentration of the solution in vacuo.

Thin layer chromatography (TLC) was performed on Merck TLC-plates (Silica gel 60 F₂₅₄) and UV visualized the spots. Flash chromatography was performed on a Combi Flash® Companion™ using RediSep™ normal-phase flash columns. Typical solvents used for flash chromatography were mixtures of chloroform/methanol, dichloromethane/methanol, heptane/ethyl acetate, chloroform/methanol/ammonia (aq.) and dichlorormethane/methanol/NH₃ (aq.). SCX ion exchange columns were performed on Isolute® columns. Chromatography through ion exchange columns were typically performed in solvents such a methanol.

Preparative chromatography was run on a Waters autopurification HPLC with a diode array detector. Column: XTerra MS C8, 19×300 mm, 10 μm. Narrow gradients with MeCN/(95:5 0.1M NH₄OAc:MeCN) were used at a flow rate of 20 ml/min. Alternatively, purification was achieved on a semi preparative Shimadzu LC-8A HPLC with a Shimadzu SPD-10A UV-vis.-detector equipped with a Waters Symmetry® column (C18, 5 μm, 100 mm×19 mm). Narrow gradients with MeCN/0.1% trifluoroacetic acid in MilliQ Water were used at a flow rate of 10 ml/min.

The formation of hydrochloride salts of the final products were typically performed in solvents or solvents mixtures such as diethyl ether, tetrahydrofuran, dichloromethane/toluene, dichloromethane/methanol, followed by addition of 1M hydrogen chloride in diethyl ether.

The following abbreviations have been used:

aq. aqueous;
BINAP 2,2′-bis(diphenylphosphino)-1,1′-binaphthyl
CHCl₃. chloroform
CDCl₃ deuterated chloroform
CD₃OD deuterated methanol
CH₂Cl₂ dichloromethane
Cs₂CO₃ caesium carbonate

### DMF N-N-dimethylformamide;

DMFDMA dimethylformamide dimethylacetal;
DMSO dimethyl sulphoxide;
dppf 1,1′-bis(diphenylphosphino)ferrocene;
EDC 1-ethyl-3-(3-dimethylaminopropyl)carbodiimide;
EDTA ethylenediaminetetraacetic acid;
ether diethyl ether;
EtOAc ethyl acetate;
EtOH ethanol
H₂ hydrogen gas
HCOOH acetic acid
HCl hydrochloride
HOAc acetic acid
HOBt 1-hydroxybenzotriazole;
(i-Pr)₂NEt N-N-diisopropylethylamine;
MeCN acetonitrile;
MeOH methanol;
Me₃SnCl trimethyltin chloride;
MgSO₄ magnesium suphate;
NaCl sodium chlodride;
NaBH₃CN sodium cyanoborohydride;
NaHCO₃ sodium bicarbonate;
NaOMe sodium methoxide;
Na₂SO₄ sodium sulphate;
n-BuOH n-butanol;
NH₃ ammonia;
NH₄OAc ammonium acetate;
NH₄OH ammonium hydroxide;
Pd/C palladium on carbon;
Pd(PPh₃)₂Cl₂ bis(triphenylphosphine)palladium dichloride;
Pd(t-Bu₃P)₂ bis(tri-tert-butylphosphine)palladium;
Pd₂(dba)₃ tris(dibenzylideneacetone)dipalladium;
Pd(OAc)₂ palladium diacetate;
r.t. or RT room temperature;
Selectfluor N-fluoro-N′-chloromethyl-triethylenediamine-bis(tetrafluoroborate);
t-BuLi tert-butyllithium
TEA triethylamine;
THF tetrahydrofuran;
X-Phos 2-dicyclohexylphosphino-2′,4′,6′-triiso-propyl-1,1′-biphenyl.

Starting materials used were either available from commercial sources or prepared according to literature procedures and had experimental data in accordance with those reported. The following is an example of a starting material that was prepared: (4-Bromophenyl)(pyridin-2-yl)methanone: Bruce, R. B. et al., J. Med. Chem. 1968, 5, 1031-1034.

Compounds have been named either using ACD/Name, version 8.08, software from Advanced Chemistry Development, Inc. (ACD/Labs), Toronto ON, Canada, www.acdlabs.com, 2004 or using Openeye lexichem version 1.4 (Copyright © 1997-2006 OpenEye Scientific Software, Santa Fe, N. Mex.) to generate the IUPAC name.

In the following general methods A to C, the groups R¹, R² and R³ are used independently to indicate the diversity of substitution within each structure. The identity of R¹, R² and R³ will be clear to a person skilled in the art based on the starting materials and intermediates for each specific example. For instance in Example 15, which refers to General method C, C1 is 5-fluoro-4-[2-trifluoromethyl-1-isopropyl-1H-imidazol-5-yl]pyrimidin-2-amine such that R¹ is —CF₃ and C2 is 4-bromophenylsulphonylmethane such that R² is -sulphonylmethane para- to the halogen.

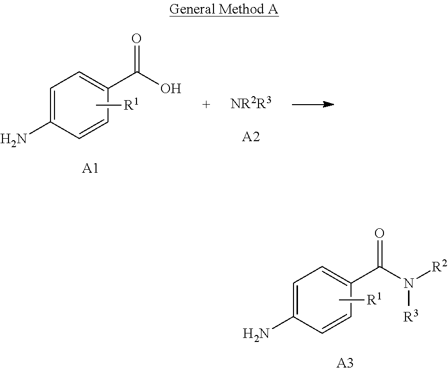

(i-Pr)₂NEt (2.1 equiv.), HOBt (1.05 equiv.), EDC hydrochloride (1.05 equiv.) and the amine A2 (1.05 equiv.) were added to a stirred solution of the benzoic acid A1 (1.0 equiv.) in anhydrous DMF at r.t. After 15 h, the reaction mixture was poured onto water and extracted with EtOAc. The organic layer was washed with water and brine, dried (Na₂SO₄), filtered and evaporated in vacuo to afford the crude product, which was used in the next step without further purification.

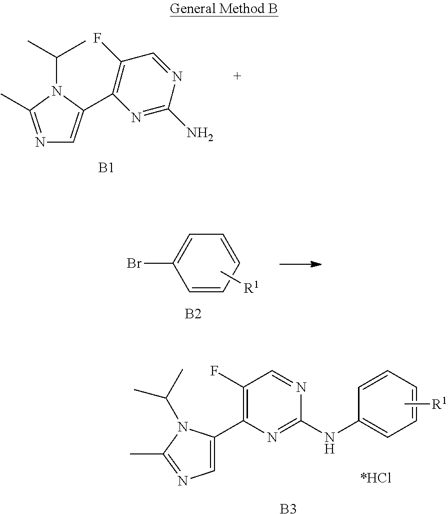

B1 (1.0 equiv.), B2 (0.85-1.24 equiv.) and sodium tert-butoxide (1.34-1.46 equiv.) were mixed in 1,4-dioxane and the mixture was flushed with argon for 5-10 minutes before Pd(OAc)₂ (0.04-0.082 equiv.) and Pd(t-Bu₃P)₂ (0.044-0.06 equiv.) were added. The mixture was flushed with argon then heated in a sealed tube at +110° C.-+120° C. until the reaction was complete (as monitored by TLC or LC-MS). If the reaction was not complete after 24 h more Pd(OAc)₂, Pd(t-Bu₃P)₂ and sodium tert-butoxide were added. The solvent was removed in vacuo and the residue was partitioned between CH₂Cl₂ and water. After extraction the organic layer was dried (Na₂SO₄), filtered and evaporated. The crude of the free base was purified using preparative HPLC. MeCN was evaporated in vacuo and the aqueous phase was extracted with CH₂Cl₂. The organic phase was washed with water at pH 9 (diluted NaHCO₃ solution), dried (Na₂SO₄), filtered and evaporated. The residue was dissolved in CH₂Cl₂ and the HCl-adduct of the product was precipitated from the solution by addition of 0.1M HCl in ether (2-3 equiv. HCl). The solvent was evaporated and the residue was dissolved in water and freeze dried.

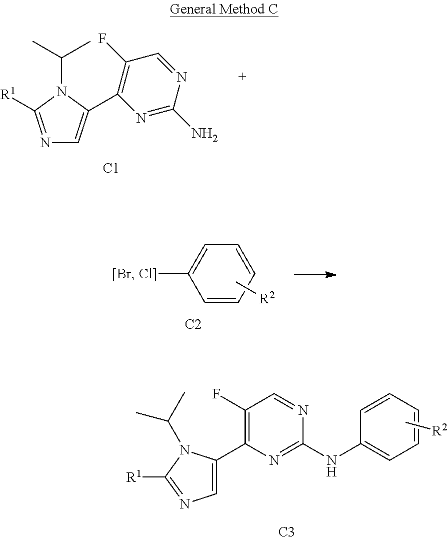

C1 (1.01-1.27 equiv.), C2 (1.0 equiv.) and Cs₂CO₃ (1.66-2.02 equiv.) were mixed in anhydrous 1,4-dioxane and the mixture was flushed with argon for 5 minutes before Pd₂(dba)₃ (0.05-0.08 equiv.) and X-Phos (0.10-0.16 equiv.) were added. The mixture was flushed with argon, then heated in a sealed tube at +90-100° C. until the reaction was complete. The solvent was removed in vacuo and the residue was taken up in CH₂Cl₂ and washed with diluted NaHCO₃ (aq.). The organic layer was dried (Na₂SO₄), filtered and evaporated. The crude of the base product was purified using preparative HPLC.

### EXAMPLES

Below follows a number of non-limiting examples of compounds of the invention.

### Example 1

### (4-{[5-Fluoro-4-(1-isopropyl-2-methyl-1H-imidazol-5-yl)pyrimidin-2-yl]amino}phenyl)(phenyl)methanone hydrochloride

### Example 1(a)

### (2Z)-3-(Dimethylamino)-2-fluoro-1-(1-isopropyl-2-methyl-1H-imidazol-5-yl)prop-2-en-1-one

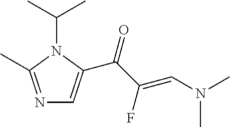

Selectfluor (1.32 g, 3.72 mmol) was added to a cooled (−70° C.) solution of (2E)-3-(dimethylamino)-1-(1-isopropyl-2-methyl-1H-imidazol-5-yl)prop-2-en-1-one (described in WO 03/076436) (0.634 g, 2.86 mmol) in MeOH (10 mL) under nitrogen atmosphere. The reaction mixture was stirred at −70° C. for 25 minutes, then stirred at r.t. for 3 h. Ammonia (32% aq., 3 mL) was added and the two layers were separated. After extraction with CH₂Cl₂, the combined organic phases were washed with saturated NaCl (aq.), dried (Na₂SO₄), filtered and evaporated in vacuo. The crude product was purified by flash chromatography (CH₂Cl₂/MeOH gradient; 0 to 10% MeOH) to give the title compound (0.311 g, 45%).

¹H NMR (CDCl₃) δ ppm 7.34 (s, 1H), 6.85 (d, J=27.6 Hz, 1H), 5.05-4.94 (m, 1H), 3.11 (s, 3H), 3.11 (s, 3H), 2.52 (s, 3H), 1.53 (d, J=7.0 Hz, 6H); MS (ESI) m/z 240 (M+1).

### Example 1(b)

### 5-Fluoro-4-(1-isopropyl-2-methyl-1H-imidazol-5-yl)pyrimidin-2-amine

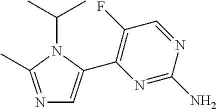

A reaction mixture of (2Z)-3-(dimethylamino)-2-fluoro-1-(1-isopropyl-2-methyl-1H-imidazol-5-yl)prop-2-en-1-one (0.170 g, 0.71 mmol, obtained from Example 1(a)), guanidine hydrochloride (0.170 g, 1.78 mmol) and sodium methoxide (0.154 g, 2.84 mmol) in 1-butanol was heated in a microwave reactor for 10 minutes at +140° C. under argon or nitrogen atmosphere. The mixture was filtered and the filtered mixture was rinsed with CH₂Cl₂. The solvent was evaporated in vacuo and the crude product was purified using flash column chromatography (EtOAc/MeOH-gradient; 0-10% MeOH) to give the title compound (0.119 g, 71%).

¹H NMR (CDCl₃) δ ppm 8.16 (d, J=3.3 Hz, 1H), 7.55 (d, J=3.8 Hz, 1H), 5.49-5.34 (m, 1H), 4.86 (br s, 2H), 2.60 (s, 3H), 1.57 (d, J=7.1 Hz, 6H); MS (ESI) m/z 236 (M+1).

### Example 1(c)

### (4-{[5-Fluoro-4-(1-isopropyl-2-methyl-1H-imidazol-5-yl)pyrimidin-2-yl]amino}phenyl)(phenyl)methanone hydrochloride

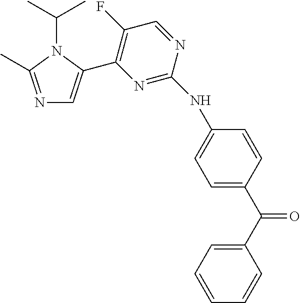

The title compound was prepared in accordance with the general method B using 5-fluoro-4-(1-isopropyl-2-methyl-1H-imidazol-5-yl)pyrimidin-2-amine (50 mg, 0.21 mmol, obtained from Example 1(b)), 4-bromobenzophenone (53.4 mg, 0.20 mmol), sodium tert-butoxide (27.4 mg, 0.28 mmol), Pd(OAc)₂ (3.3 mg, 0.015 mmol) and Pd(t-Bu₃P)₂ (5.1 mg, 0.010 mmol) to give the title compound (28 mg, 32%).

¹H NMR (DMSO-d₆) δ ppm 10.35 (s, 1H), 8.87 (d, J=2.0 Hz, 1H), 8.05 (s, 1H), 7.88 (d, J=8.8 Hz, 2H), 7.75 (d, J=8.6 Hz, 2H), 7.72-7.62 (m, 3H), 7.55 (t, J=7.5 Hz, 2H), 5.36-5.20 (m, 1H), 2.77 (s, 3H), 1.51 (d, J=6.8 Hz, 6H): MS (ESI) m/z 416 (M+1).

### Example 2

### (4-{[5-Fluoro-4-(1-isopropyl-2-methyl-1H-imidazol-5-yl)pyrimidin-2-yl]amino}phenyl)(pyridin-2-yl)methanone hydrochloride

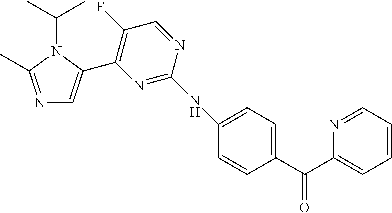

The title compound was prepared in accordance with the general method B but using a 70 h reaction time. Starting from 5-fluoro-4-(1-isopropyl-2-methyl-1H-imidazol-5-yl)pyrimidin-2-amine (obtained from Example 1(b)) (47.7 mg, 0.203 mmol), (4-bromophenyl)(pyridin-2-yl)methanone (Bruce, R. B., et al., J. Med. Chem., 1968, 5, 1031-1034) (53.8 mg, 0.205 mmol), sodium tert-butoxide (27.8 mg, 0.289 mmol), Pd(OAc)₂ (2.5 mg, 0.011 mmol) and Pd(t-Bu₃P)₂ (6.5 mg, 0.013 mmol), the title compound (17 mg, 17%) was obtained as a solid.

¹H NMR (DMSO-d₆, 600 MHz) δ ppm 10.41 (s, 1H), 8.90 (d, J=1.4 Hz, 1H), 8.71 (d, J=4.6 Hz, 1H), 8.13 (d, J=1.4 Hz, 1H), 8.08-8.03 (m, 1H), 8.00 (d, J=8.8 Hz, 2H), 7.93 (d, J=7.7 Hz, 1H), 7.86 (d, J=8.8 Hz, 1H), 7.68-7.63 (m, 1H), 5.31-5.21 (m, 1H), 2.81 (s, 3H), 1.52 (d, J=6.8 Hz, 6H); MS (ESI) m/z 417 (M+1).

### Example 3

### (4-{[5-Fluoro-4-(1-isopropyl-2-methyl-1H-imidazol-5-yl)pyrimidin-2-yl]amino}phenyl)(pyridin-3-yl)methanone hydrochloride

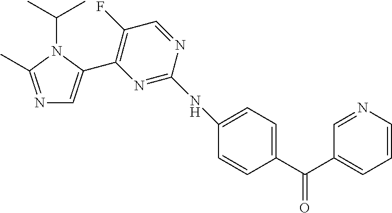

The title compound was prepared in accordance with the general method B using 5-fluoro-4-(1-isopropyl-2-methyl-1H-imidazol-5-yl)pyrimidin-2-amine (obtained from Example 1(b)) (59 mg, 0.25 mmol), (4-bromophenyl)(pyridin-3-yl)methanone (Bäckvall, J.-E., et al., J. Org. Chem., 1981, 46, 3479-3483) (56 mg, 0.213 mmol), sodium tert-butoxide (33.8 mg, 0.35 mmol), Pd(OAc)₂ (2.9 mg, 0.013 mmol) and Pd(t-Bu₃P)₂ (6.4 mg, 0.012 mmol) to give the title compound (35 mg, 34%) as a solid.

¹H NMR (DMSO-d₆) δ ppm 10.45 (s, 1H), 8.91 (d, J=2.0 Hz, 1H), 8.89 (d, J=1.3 Hz, 1H), 8.85 (d, J=1.3 Hz, 1H), 8.21-8.15 (m, 1H), 8.13 (d, J=1.8 Hz, 1H), 7.91 (d, J=8.8 Hz, 2H), 7.79 (d, J=8.8 Hz, 2H), 7.67 (dd, J=7.7, 5.1 Hz, 1H), 5.32-5.18 (m, 1H), 2.81 (s, 3H), 1.52 (d, J=7.0 Hz, 6H); MS (ESI) m/z 417 (M+1).

### Example 4

### 5-Fluoro-4-(1-isopropyl-2-methyl-1H-imidazol-5-yl)-N-{3-methyl-4-[(4-methylpiperazin-1-yl)sulfonyl]phenyl}pyrimidin-2-amine hydrochloride

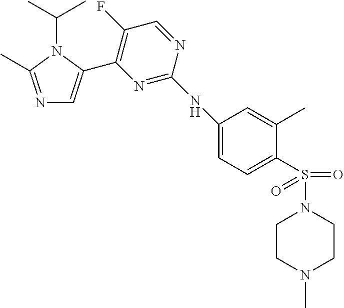

The title compound was prepared in accordance with the general method B using 5-fluoro-4-(1-isopropyl-2-methyl-1H-imidazol-5-yl)pyrimidin-2-amine (obtained from Example 1(b)) (40 mg, 0.17 mmol), 1-[(4-bromo-2-methylphenyl)sulfonyl]-4-methylpiperazine (described in WO 2003004472) (56 mg, 0.168 mmol), sodium tert-butoxide (22.7 mg, 0.236 mmol), Pd(OAc)₂ (3.0 mg, 0.013 mmol) and Pd(t-Bu₃P)₂ (5.0 mg, 0.010 mmol) to give the title compound (36 mg, 38%) as a solid.

¹H NMR (D₂O) δ ppm 8.52 (d, J=2.0 Hz, 1H), 7.82 (d, J=2.0 Hz, 1H), 7.73 (d, J=8.8 Hz, 1H), 7.55 (dd, J=8.8, 2.0 Hz, 1H), 7.42 (d, J=1.3 Hz, 1H), 5.43-5.29 (m, 1H), 3.83 (d, J=12.9 Hz, 2H), 3.59 (d, J=11.9 Hz, 2H), 3.26-3.03 (m, 4H), 2.92 (s, 3H), 2.81 (s, 3H), 2.47 (s, 3H), 1.51 (d, J=7.1 Hz, 6H); MS (ESI) m/z 488 (M+1).

### Example 5

### 5-Fluoro-4-(1-isopropyl-2-methyl-1H-imidazol-5-yl)-N-{2-methyl-4-[(4-methylpiperazin-1-yl)sulfonyl]phenyl}pyrimidin-2-amine hydrochloride

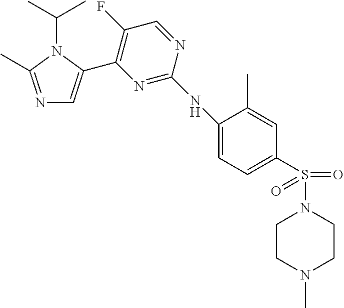

The title compound was prepared in accordance with the general method B using 5-fluoro-4-(1-isopropyl-2-methyl-1H-imidazol-5-yl)pyrimidin-2-amine (obtained from Example 1(b)) (59 mg, 0.25 mmol), 1-[(4-bromo-3-methylphenyl)sulfonyl]-4-methylpiperazine (described in WO 2003004472) (46.4 mg, 0.139 mmol), sodium tert-butoxide (21.7 mg, 0.23 mmol), Pd(OAc)₂ (2.1 mg, 0.009 mmol) and Pd(t-Bu₃P)₂ (5.0 mg, 0.010 mmol) to give the title compound (17 mg, 18%) as a solid.

¹H NMR (CD₃OD) δ ppm 8.60 (d, J=2.3 Hz, 1H), 7.98-7.89 (m, 2H), 7.73 (d, J=1.8 Hz, 1H), 7.66 (dd, J=8.3, 2.3 Hz, 1H), 5.49-5.35 (m, 1H), 4.33-3.35 (m, 8H), 2.90 (s, 3H), 2.80 (s, 3H), 2.41 (s, 3H), 1.49 (d, J=7.1 Hz, 6H); MS (ESI) m/z 488 (M+1).

### Example 6

### 5-Fluoro-4-(1-isopropyl-2-methyl-1H-imidazol-5-yl)-N-[4-[(4-methylpiperazin-1-yl)sulfonyl]-2-(trifluoromethyl)phenyl]pyrimidin-2-amine hydrochloride

### Example 6(a)

### 1-{[4-Bromo-3-(trifluoromethyl)phenyl]sulfonyl}-4-methylpiperazine

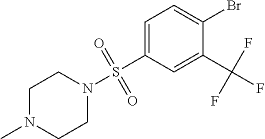

Triethylamine (1.39 mL, 10.0 mmol) was added dropwise to a solution of N-methylpiperazine (1.0 g, 10.1 mmol) and 4-bromo-3-(trifluoromethyl)benzenesulphonyl chloride (3.23 g, 10.0 mmol) in CH₂Cl₂ (50 mL). The resulting mixture was stirred at r.t. for 30 minutes. Saturated NaHCO₃ (aq., 30 mL) was added. The solution was extracted with CH₂Cl₂ and the organic layers were dried (MgSO₄), filtered and evaporated in vacuo to give the title compound (3.62 g, 94%) as a solid. This crude product was used in the next step without further purification.

¹H NMR (CDCl₃) δ ppm 8.01 (d, J=2.0 Hz, 1H), 7.89 (d, J=8.3 Hz, 1H), 7.74 (dd, J=8.3, 2.0 Hz, 1H), 3.12 (s, 4H), 2.57 (s, 4H), 2.33 (s, 3H); MS (ESI) m/z 387, 389 (M+1).

### Example 6(b)

### 5-Fluoro-4-(1-isopropyl-2-methyl-1H-imidazol-5-yl)-N-[4-[(4-methylpiperazin-1-yl)sulfonyl]-2-(trifluoromethyl)phenyl]pyrimidin-2-amine hydrochloride

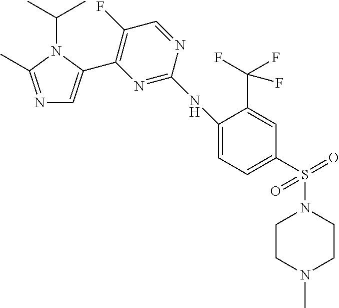

The title compound was prepared in accordance with the general procedure B but with the following modifications. After heating at +120° C. for 95 minutes, Pd(OAc)₂ (2 mg), Pd(t-Bu₃P)₂ (4.5 mg) and sodium tert-butoxide (15 mg) were added. Starting from 5-fluoro-4-(1-isopropyl-2-methyl-1H-imidazol-5-yl)pyrimidin-2-amine (obtained from Example 1(b)) (47 mg, 0.20 mmol), 1-{[4-bromo-3-(trifluoromethyl)phenyl]sulfonyl}-4-methylpiperazine (77 mg, 0.199 mmol, obtained from Example 6(a)), sodium tert-butoxide (26.6 mg, 0.277 mmol), Pd(OAc)₂ (2.0 mg, 0.009 mmol) and Pd(t-Bu₃P)₂ (5.1 mg, 0.010 mmol), the title compound (38 mg, 35%) was obtained as a solid.

¹H NMR (D₂O) δ ppm 8.51 (d, J=2.3 Hz, 1H), 8.21 (s, 1H), 8.13-8.04 (m, 2H) 7.70 (d, J=2.0 Hz, 1H), 5.22-5.09 (m, 1H), 3.77-2.99 (m, 8H), 2.82 (s, 3H), 2.68 (s, 3H), 1.37 (d, J=6.8 Hz, 6H); MS (ESI) m/z 542 (M+1).

### Example 7

### 5-Fluoro-4-(1-isopropyl-2-methyl-1H-imidazol-5-yl)-N-[4-[(4-methylpiperazin-1-yl)sulfonyl]-3-(trifluoromethoxy)phenyl]pyrimidin-2-amine hydrochloride

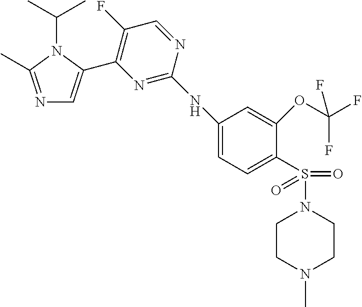

The title compound was prepared in accordance with the general method B using 5-fluoro-4-(1-isopropyl-2-methyl-1H-imidazol-5-yl)pyrimidin-2-amine (obtained from Example 1(b)) (38.5 mg, 0.164 mmol), 1-{[4-bromo-2-(trifluoromethoxy)phenyl]sulfonyl}-4-methylpiperazine (described in WO 2003004472) (86 mg, 0.203 mmol), sodium tert-butoxide (22 mg, 0.229 mmol), Pd(OAc)₂ (3.0 mg, 0.013 mmol) and Pd(t-Bu₃P)₂ (4.0 mg, 0.008 mmol) to give the title compound (16 mg, 16%) as a solid.

¹H NMR (D₂O) δ ppm 8.58 (d, J=1.8 Hz, 1H), 7.87 (br s, 1H), 7.82 (d, J=8.8 Hz, 1H), 7.78 (d, J=1.5 Hz, 1H), 7.59 (dd, J=8.8, 2.0 Hz, 1H), 5.31-5.17 (m, 1H), 4.32-2.98 (m, 8H), 2.91 (s, 3H), 2.79 (s, 3H), 1.51 (d, J=7.1 Hz, 6H); MS (ESI) m/z 558 (M+1).

### Example 8

### 5-Fluoro-4-(1-isopropyl-2-methyl-1H-imidazol-5-yl)-N-[4-[(4-methylpiperazin-1-yl)carbonyl]-3-(methylsulfonyl)phenyl]pyrimidin-2-amine hydrochloride

### Example 8(a)

### 1-[4-Bromo-2-(methylsulfonyl)benzoyl]-4-methylpiperazine

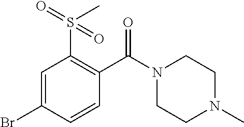

Thionyl chloride (5 mL) was added to 4-bromo-2-(methylsulfonyl)benzoic acid (0.50 g, 1.67 mmol). After addition of 1 drop of anhydrous DMF, the reaction mixture was refluxed for 30 minutes under an atmosphere of nitrogen. The solvent was evaporated in vacuo and the residue was dissolved in CH₂Cl₂ (until a clear solution was obtained). N-Methylpiperazine (0.195 mL, 1.75 mmol) was added dropwise followed by addition of triethylamine (0.243 mL, 1.75 mmol). The reaction mixture was stirred at r.t. for 15 minutes before it was diluted with CH₂Cl₂, washed with saturated NaHCO₃ (aq.), water, dried (Na₂SO₄), filtered and concentrated in vacuo to give the title compound in quantitative yield. The isolated material was used in the next step without further purification.

¹H NMR (DMSO-d₆, Signals corresponding to 3 protons were overlapping with the solvents) δ ppm 8.06 (d, J=2.0 Hz, 1H), 8.01 (dd, J=8.3, 2.0 Hz, 1H), 7.46 (d, J=8.0 Hz, 1H), 3.65-3.53 (m, 2H), 3.19-3.00 (m, 2H), 2.43-2.33 (m, 2H), 2.33-2.21 (m, 2H), 2.19 (s, 3H); MS (ESI) m/z 361, 363 (M+1).

### Example 8(b)

### 5-Fluoro-4-(1-isopropyl-2-methyl-1H-imidazol-5-yl)-N-[4-[(4-methylpiperazin-1-yl)carbonyl]-3-(methylsulfonyl)phenyl]pyrimidin-2-amine hydrochloride

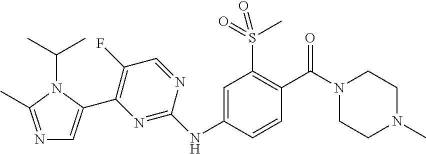

Anhydrous 1,4-dioxane (2 mL) was added to 5-fluoro-4-(1-isopropyl-2-methyl-1H-imidazol-5-yl)pyrimidin-2-amine (obtained from Example 1(b)) (52 mg, 0.22 mmol), 1-[4-bromo-2-(methylsulfonyl)benzoyl]-4-methylpiperazine (Bruce, R. B., et al. J. Med. Chem., 1968, 5, 1031-1034) (71.0 mg, 0.197 mmol, obtained from Example 8(a)) and sodium tert-butoxide (30.9 mg, 0.32 mmol). The mixture was purged with argon and Pd(OAc)₂ (2 mg, 0.009 mmol), Pd(t-Bu₃P)₂ (6.1 mg, 0.012 mmol) were added followed by another argon purge. The mixture was heated in a sealed tube at 120° C. After stirring overnight Pd(t-Bu₃P)₂ (12.4 mg, 0.024 mmol) was added and after another 24 h the following reagents were added; Cs₂CO₃ (107 mg, 0.33 mmol), X-Phos (11.3 mg, 0.024 mmol) and Pd₂(dba)₃ (11.1 mg, 0.012 mmol). The resulting mixture was heated in an oil bath for +90° C. for 20 h. The mixture was filtered through diatomaceous earth and washed with EtOAc. The organic phase was washed with water, dried (Na₂SO₄), filtered and evaporated in vacuo. The residue was purified by flash chromatography (MeCN/5% TEA in MeCN gradient; 0 to 5% TEA in MeCN). The product-containing fractions were pooled together and evaporated in vacuo. The residue was dissolved in CH₂Cl₂ and the organic phase was washed with EDTA (aq.) at pH 1. The EDTA (aq.) phase was neutralized (pH 7) with NaHCO₃ (aq.) and the product was extracted with CH₂Cl₂. The organic phase was dried (Na₂SO₄), filtered and evaporated in vacuo. The residue was dissolved in CH₂Cl₂/ether (1:1, 10 mL) and the title compound precipitated by dropwise addition of 1M HCl in ether (2.0 equiv.). The precipitate was collected by filtration, rinsed with CH₂Cl₂, dissolved in water and freeze dried to give the title compound (96 mg, 74%) as a solid.

¹H NMR (DMSO-d₆) δ ppm 11.47-11.25 (m, 1H), 10.41 (s, 1H), 8.90 (d, J=1.5 Hz, 1H), 8.30 (s, 1H), 8.21 (d, J=8.5 Hz, 1H), 8.12 (s, 1H), 7.52 (d, J=8.0 Hz, 1H), 5.29-5.12 (m, 1H), 4.64-4.48 (m, 1H), 3.62-3.04 (m, 6H), 2.84-2.71 (m, 7H), 2.76 (br s, 4H), 1.52 (d, J=7.0 Hz, 6H); MS (ESI) m/z 516 (M+1).

### Example 9

### 5-{[5-Fluoro-4-(1-isopropyl-2-methyl-1H-imidazol-5-yl)pyrimidin-2-yl]amino}-2-[(4-methylpiperazin-1-yl)carbonyl]benzonitrile hydrochloride

### Example 9(a)

### 1-(4-Chloro-2-iodobenzoyl)-4-methylpiperazine

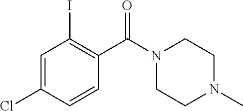

The title compound was prepared in accordance with the general method of Example 8 (a) with the exception that the reaction mixture was stirred in thionyl chloride under reflux for 60 minutes, and the reaction with the amine was stirred at r.t. overnight. Using 4-chloro-2-iodobenzoic acid (0.523 g, 1.85 mmol) and after purification with flash chromatography (CHCl₃/MeOH gradient; 0 to 5% MeOH), the title compound (0.415 g, 61%) was obtained as a solid.

¹H NMR (DMSO-d₆) δ ppm 7.97 (d, J=2.0 Hz, 1H), 7.54 (dd, J=8.3, 2.0 Hz, 1H), 7.26 (d, J=8.3 Hz, 1H), 3.69-3.53 (m, 2H), 3.08 (t, J=5.0 Hz, 2H), 2.38 (t, J=5.1 Hz, 2H), 2.36-2.21 (m, 2H), 2.19 (s, 3H); MS (ESI) m/z 365 (M+1).

### Example 9(b)

### 5-Chloro-2-[(4-methylpiperazin-1-yl)carbonyl]benzonitrile

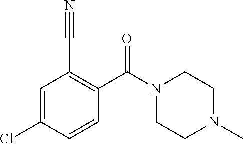

1-(4-Chloro-2-iodobenzoyl)-4-methylpiperazine (400 mg, 1.70 mmol, obtained from Example 9(a)), zinc acetate (17.2 mg, 0.079 mmol), zinc cyanide (109 mg, 0.928 mmol), zinc dust (8.0 mg, 0.122 mmol), Pd₂(dba)₃ (44 mg, 0.048 mmol) and dppf (117 mg, 0.211 mmol) were mixed in anhydrous 1,4-dioxane (1.5 mL) and flushed with argon. The mixture was heated in a sealed tube at +90-+95° C. for 45 minutes. The reaction mixture was filtered through diatomaceous earth and rinsed with EtOAc. The organic phase was washed with saturated NaHCO₃ (aq.), dried (Na₂SO₄), filtered and evaporated in vacuo. The crude product was purified by flash chromatography (CH₂Cl₂/MeOH-gradient; 0 to 5% MeOH) to give the title compound (280 mg, 62%).

¹H NMR (DMSO-d₆) δ ppm 8.16 (d, J=2.0 Hz, 1H), 7.87 (dd, J=8.3, 2.0 Hz, 1H), 7.59 (d, J=8.3 Hz, 1H), 3.71-3.59 (m, 2H), 3.24-3.14 (m, 2H), 2.43-2.32 (m, 2H), 2.32-2.22 (m, 2H), 2.19 (s, 3H); MS (ESI) m/z 264 (M+1).

### Example 9(c)

### 5-{[5-Fluoro-4-(1-isopropyl-2-methyl-1H-imidazol-5-yl)pyrimidin-2-yl]amino}-2-[(4-methylpiperazin-1-yl)carbonyl]benzonitrile hydrochloride

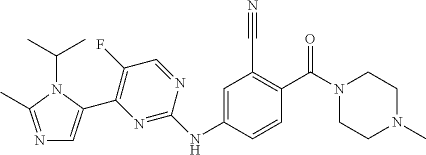

The title compound was prepared in accordance with the general method C using 5-fluoro-4-(1-isopropyl-2-methyl-1H-imidazol-5-yl)pyrimidin-2-amine (obtained from Example 1(b)) (53 mg, 0.225 mmol), 5-chloro-2-[(4-methylpiperazin-1-yl)carbonyl]benzonitrile (55 mg, 0.209 mmol, obtained from Example 9(b)), Cs₂CO₃ (114 mg, 0.350 mmol), Pd₂(dba)₃ (11.6 mg, 0.013 mmol) and X-Phos (10.6 mg, 0.022 mmol) to give the title compound (70 mg, 58%) as a solid. The hydrochloride was prepared in accordance with the general method D.

¹H NMR (DMSO-d₆) δ ppm 11.79 (br s, 1H), 10.54 (s, 1H), 8.91 (d, J=1.76 Hz, 1H), 8.29 (d, J=2.01 Hz, 1H), 8.11 (d, J=1.83 Hz, 1H), 8.02 (dd, J=8.53, 2.26 Hz, 1H), 7.62 (d, J=8.53 Hz, 1H), 5.26 (s, 1H), 4.57 (br s, 1H), 3.75-2.95 (m, 8H), 2.82 (s, 3H), 2.77 (s, 3H), 1.52 (d, J=7.03 Hz, 6H); MS (ESI) m/z 463 (M+1).

### Example 10

### N-{3-Chloro-4-[(4-methylpiperazin-1-yl)carbonyl]phenyl}-5-fluoro-4-(1-isopropyl-2-methyl-1H-imidazol-5-yl)pyrimidin-2-amine hydrochloride

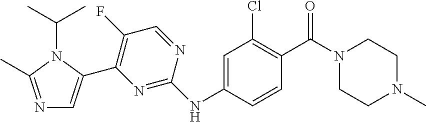

The title compound was prepared in accordance with the general method C using 5-fluoro-4-(1-isopropyl-2-methyl-1H-imidazol-5-yl)pyrimidin-2-amine (obtained from Example 1(b)) (49.4 mg, 0.211 mmol), 1-(2,4-dichlorobenzoyl)-4-methylpiperazine (Prasad, R. N., et al., J. Med. Chem., 1968, 6, 1144-1150) (57 mg, 0.209 mmol), Cs₂CO₃ (113 mg, 0.347 mmol), Pd₂(dba)₃ (9.9 mg, 0.011 mmol) and X-Phos (10.5 mg, 0.022 mmol) to give the title compound (50 mg, 44%) as a yellow solid. The hydrochloride was prepared in accordance with the general method B.

¹H NMR (DMSO-d₆) δ ppm 11.78 (br s, 1H), 10.31 (s, 1H), 8.88 (d, J=1.8 Hz, 1H), 8.12 (d, J=2.0 Hz, 1H), 7.94 (br s, 1H), 7.71 (dd, J=8.3, 2.0 Hz, 1H), 7.38 (br s, 1H), 5.30-5.17 (m, 1H), 4.57 (d, J=13.1 Hz, 1H), 4.20-2.89 (m, 8H), 2.83 (s, 3H), 2.76 (s, 3H), 1.51 (d, J=7.1 Hz, 6H); MS (ESI) m/z 472 (M+1).

### Example 11

### 5-Fluoro-4-(1-isopropyl-2-methyl-1H-imidazol-5-yl)-N-{3-methoxy-4-[(4-methylpiperazin-1-yl)carbonyl]phenyl}pyrimidin-2-amine hydrochloride

### Example 11(a)

### 1-(4-Chloro-2-methoxybenzoyl)-4-methylpiperazine

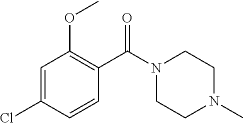

The title compound was prepared in accordance with the general method of Example 8(a) using 4-chloro-2-methoxybenzoic acid (0.501 g, 2.68 mmol) to give the title compound in quantitative yield. This crude product was used in the next step without further purification

¹H NMR (DMSO-d₆) δ ppm 7.18 (d, J=8.0 Hz, 1H), 7.17 (d, J=2.0 Hz, 1H), 7.05 (dd, J=8.0, 1.8 Hz, 1H), 3.81 (s, 3H), 3.67-3.51 (m, 2H), 3.14-3.04 (m, 2H), 2.38-2.27 (m, 2H), 2.27-2.19 (m, 2H), 2.18 (s, 3H).

### Example 11(b)

### 5-Fluoro-4-(1-isopropyl-2-methyl-1H-imidazol-5-yl)-N-{3-methoxy-4-[(4-methylpiperazin-1-yl)carbonyl]phenyl}pyrimidin-2-amine hydrochloride

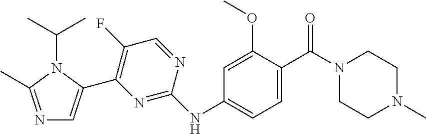

The title compound was prepared in accordance with the general method C using 5-fluoro-4-(1-isopropyl-2-methyl-1H-imidazol-5-yl)pyrimidin-2-amine (obtained from Example 1(b)) (49.8 mg, 0.212 mmol), 1-(4-chloro-2-methoxybenzoyl)-4-methylpiperazine (45 mg, 0.167 mmol), Cs₂CO₃ (110 mg, 0.338 mmol), Pd₂(dba)₃ (9.3 mg, 0.010 mmol) and X-Phos (11.3 mg, 0.024 mmol). The hydrochloride was prepared in accordance with the general method D to give the title compound (60 mg, 53%) as a solid.

¹H NMR (DMSO-d₆) δ ppm 11.38 (br s, 1H), 10.06 (s, 1H), 8.85 (d, J=2.0 Hz, 1H), 8.12 (d, J=1.8 Hz, 1H), 7.49 (dd, J=8.3, 1.8 Hz, 1H), 7.39 (s, 1H), 7.20 (d, J=8.3 Hz, 1H), 5.30-5.15 (m, 1H), 4.58 (d, J=13.3 Hz, 1H), 3.81 (s, 3H), 3.57-3.13 (m, 6H), 3.13-2.87 (m, 2H), 2.82 (s, 3H), 2.79 (br s, 3H), 1.51 (d, J=7.0 Hz, 6H); MS (ESI) m/z 468 (M+1).

### Example 12

### (4-{[5-Fluoro-4-(1-isopropyl-2-methyl-1H-imidazol-5-yl)pyrimidin-2-yl]amino}phenyl)(pyridin-4-yl)methanone hydrochloride

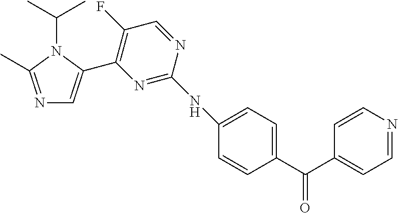

The title compound was prepared in accordance with the general method C, with the exception that the base of the product was purified by flash chromatography (EtOAc/4% MeOH in EtOAc gradient; 0 to 4% MeOH in EtOAc). The hydrochloride of the title compound was prepared in accordance with the general method D. Using 5-fluoro-4-(1-isopropyl-2-methyl-1H-imidazol-5-yl)pyrimidin-2-amine (obtained from Example 1(b)) (42.6 mg, 0.181 mmol), (4-chlorophenyl)(pyridin-4-yl)methanone (Högberg, T., et al., J. Med. Chem., 1981, 24, 1499-1507) (34.6 mg, 0.159 mmol), Cs₂CO₃ (93.0 mg, 0.285 mmol), Pd₂(dba)₃ (11.6 mg, 0.013 mmol) and X-Phos (12.6 mg, 0.026 mmol), the title compound (26 mg, 34%) was obtained as a solid.

¹H NMR (DMSO-d₆) δ ppm 10.51 (s, 1H), 8.91 (d, J=1.8 Hz, 1H), 8.88 (d, J=4.8 Hz, 2H), 8.13 (d, J=1.5 Hz, 1H), 7.92 (d, J=8.8 Hz, 2H), 7.83-7.72 (m, 4H), 5.31-5.17 (m, 1 H), 2.82 (s, 3H), 1.52 (d, J=7.0 Hz, 6H); MS (ESI) m/z 417 (M+1).

### Example 13

### 5-Fluoro-4-(1-isopropyl-2-methyl-1H-imidazol-5-yl)-N-{4-[(4-methylpiperazin-1-yl)carbonyl]phenyl}pyrimidin-2-amine hydrochloride

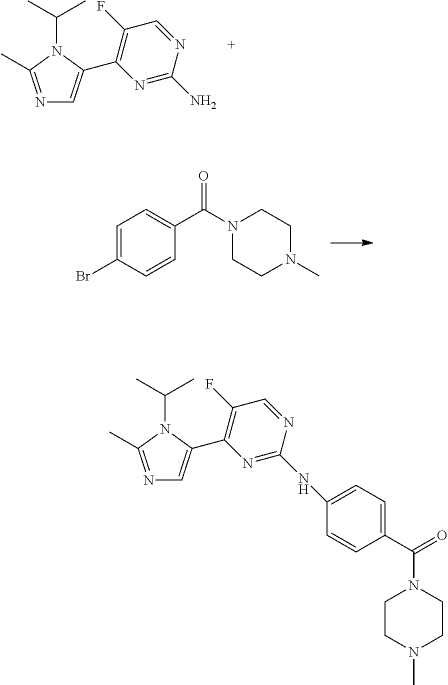

5-Fluoro-4-(1-isopropyl-2-methyl-1H-imidazol-5-yl)pyrimidin-2-amine (obtained from Example 1(b)) (64 mg, 0.272 mmol), 1-(4-bromobenzoyl)-4-methylpiperazine (described in WO 2003004472) (92 mg, 0.325 mmol), BINAP (51 mg, 0.082 mmol) and sodium tert-butoxide (31 mg, 0.323 mmol) were mixed in 1,4-dioxane (2.0 mL). The mixture was flushed with argon for 5 minutes, then Pd(OAc)₂ (9.1 mg, 0.045 mmol) was added followed by another purge with argon. The reaction mixture was heated in a sealed tube at +110° C. for 30 minutes in a microwave reactor. The solvent was evaporated in vacuo and the residue was partitioned between CH₂Cl₂ and diluted NaHCO₃ (aq.). The aqueous phase was extracted with CH₂Cl₂ and the combined organic phases were dried (Na₂SO₄), filtered and evaporated. The crude was purified using preparative HPLC. The hydrochloride was prepared in accordance with the general method D to give the title compound (43 mg, 36%) as a solid.

¹H NMR (D₂O) δ ppm 8.54 (d, J=2.0 Hz, 1H), 7.79 (d, J=2.0 Hz, 1H), 7.63 (d, J=8.8 Hz, 2H), 7.48 (d, J=8.6 Hz, 2H), 5.39-5.26 (m, 1H), 4.29-3.83 (m, 2H), 3.79-3.33 (m, 4H), 3.32-3.09 (m, 2H), 2.95 (s, 3H), 2.79 (s, 3H), 1.52 (d, J=6.8 Hz, 6H); MS (ESI) m/z 437 (M+1).

### Example 14

### 5-Fluoro-4-(1-isopropyl-2-methyl-1H-imidazol-5-yl)-N-{4-[(4-methylpiperazin-1-yl)sulfonyl]phenyl}pyrimidin-2-amine hydrochloride

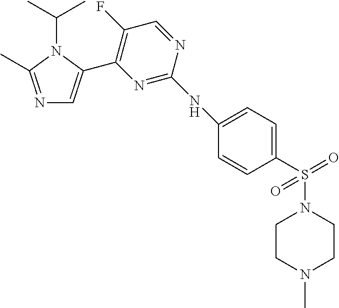

The title compound was prepared in accordance with the general method described in Example 13 with the exception that the base of the product was dissolved in CH₂Cl₂/toluene (1:1) when preparing the hydrochloride salt. Using 5-fluoro-4-(1-isopropyl-2-methyl-1H-imidazol-5-yl)pyrimidin-2-amine (obtained from Example 1(b)) (38.0 mg, 0.161 mmol), 1-[(4-bromophenyl)sulfonyl]-4-methylpiperazine (described in WO 2003004472) (78.0 mg, 0.244 mmol), BINAP (20.0 mg, 0.032 mmol), sodium tert-butoxide (20.0 mg, 0.208 mmol) and Pd(OAc)₂ (5.0 mg, 0.022 mmol) the title compound (27 mg, 31%) was obtained.

¹H NMR (DMSO-d₆) δ ppm 11.00 (br s, 1H), 10.46 (s, 1H), 8.89 (d, J=1.8 Hz, 1H), 8.09 (d, J=1.5 Hz, 1H), 7.97 (d, J=8.8 Hz, 2H), 7.70 (d, J=8.8 Hz, 2H), 5.31-5.17 (m, 1H), 3.79-3.68 (m, J=11.4 Hz, 2H), 3.42 (d, J=11.1 Hz, 2H), 3.13 (s, 2H), 2.79 (s, 3H), 2.72 (s, 3H), 2.71-2.59 (m, 2H), 1.52 (d, J=7.1 Hz, 6H); MS (ESI) m/z 474 (M+1).

### Example 15

### 5-Fluoro-4-[1-isopropyl-2-(trifluoromethyl)-1H-imidazol-5-yl]-N-[4-(methylsulfonyl)phenyl]pyrimidin-2-amine hydrochloride

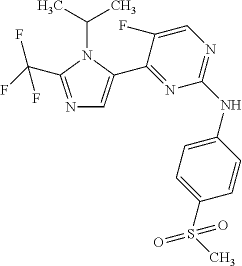

The title compound was prepared in accordance with the general method C using 5-fluoro-4-[2-trifluoromethyl-1-isopropyl-1H-imidazol-5-yl]pyrimidin-2-amine (obtained from Example 15(e)) (50 mg, 0.173 mmol), 4-bromophenyl methyl sulfone (41 mg, 0.173 mmol), Cs₂CO₃ (114 mg, 0.350 mmol), Pd₂(dba)₃ (6 mg, 0.006 mmol) and X-Phos (5.3 mg, 0.011 mmol) to give the freebase of the title compound (41 mg, 53%) as a solid. The hydrochloride was prepared in accordance with the method described within general method B.

¹H NMR (400 MHz, DMSO-d₆) δ ppm 10.35 (s, 1H) 8.85 (s, 1H) 7.76-8.04 (m, 4H) 7.54 (br. s., 1H) 4.97-5.13 (m, 1H) 3.89 (br. s., 3H) 1.47 (d, J=6.57 Hz, 6H). MS (ES) m/z 444 (M+1).

### Example 15(a)

### 2,2,2-Trifluoro-N-isopropyl-N-(5-methyl-isoxazol-4-yl)-acetamide

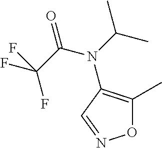

5-Methyl-4-amino-isoxazole (Reiter, L. A., J. Org. Chem. 1987, 52, 2714-2726) (0.68 g, 5.1 mmol) and acetic acid (0.61 g, 10.2 mmol) were dissolved in MeOH (20 mL). Acetone (0.56 ml, 7.6 mmol) was added and the mixture was cooled to 0-(−5)° C. and stirred for 1 h. Sodium cyanoborohydride (0.32 g, 5.1 mmol) was added to the reaction mixture at −5° C., causing weak exothermic and gas evolution. The cooling bath was removed and the mixture was stirred at r.t. for 1 h, followed by the addition of a second portion of sodium cyanoborohydride (0.1 g, 1.6 mmol). After stirring for 2 h at r.t., the mixture was filtered and the filtrate was concentrated in vacuo. The residue was dissolved in toluene and re-concentrated. The residue was dissolved in THF (10 mL) and trifluoro acetic anhydride (3.2 g, 15.3 mmol) was added. The resulting mixture was stirred overnight at r.t. then for 1 h at +50° C. The volatiles were removed in vacuo and the residue was dissolved in toluene and concentrated in vacuo to give the title compound (0.84 g, 77%) as a solid.

¹H NMR (400 MHz, CDCl₃) δ ppm 8.11 (s, 1H) 4.82-5.03 (m, 1H) 2.39 (s, 3H) 1.16 (d, J=6.82 Hz, 3H) 1.08 (d, J=6.82 Hz, 3H); MS (CI) m/z 236 (M⁺).

### Example 15(b)

### 5-Acetyl-2-trifluoromethyl-1-isopropyl-1H-imidazole

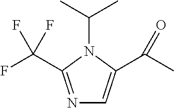

2,2,2-Trifluoro-N-isopropyl-N-(5-methyl-isoxazol-4-yl)-acetamide (2 g, 8.5 mmol, obtained from Example 15(a)) was dissolved in EtOH (30 ml), and the mixture was hydrogenated over Pd/C (10%, wet paste, 0.10 g) at 3 bar. The reaction mixture was stirred at +50° C. for 3 h. An additional amount of Pd/C (10%, wet paste, 0.15 g) was added and the mixture was continued stirring at +50° C. for 3 h. Sodium methoxide (1.38 g, 26 mmol) was added and the resulting mixture was heated at reflux for 30 h. Ammonium chloride was added to quench the reaction. The mixture was filtered through diatomaceous earth and the filtrate was evaporated in vacuo. The residue was diluted with saturated sodium bicarbonate (aq.) and extracted with EtOAc, then with CHCl₃. The combined organic layers were dried (Na₂SO₄) and concentrated in vacuo. The crude product was purified by flash chromatography (CH₂Cl₂/MeOH, 20:1) to give the title compound (1.33 g, 71%).

¹H NMR (400 MHz, CDCl₃) δ ppm 7.84 (s, 1H) 4.96 (s, 1H) 2.56 (s, 3H) 1.58 (d, J=6.82 Hz, 6H); MS (CI) m/z 220 (M⁺).

### Example 15(c)

### (2E)-3-Dimethylamino-1-(2-trifluoromethyl-1-isopropyl-1H-imidazol-5-yl)prop-2-en-1-one

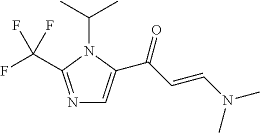

5-Acetyl-2-trifluoromethyl-1-isopropyl-1H-imidazole (1.33 g, 6 mmol, obtained from Example 15(b)) was dissolved in DMFDMA/DMF (1:1, 100 mL) and the mixture was stirred under reflux overnight. After cooling to r.t. the mixture was extracted with CH₂Cl₂. The organic phase was dried (Na₂SO₄), filtered and concentrated in vacuo. The crude product was purified by flash chromatography (CH₂Cl₂/MeOH 25:1) to give the title compound (1.6 g, 73%).

¹H NMR (400 MHz, CDCl₃) δ ppm 7.68 (d, J=12.38 Hz, 1H) 7.47 (s, 1H) 5.49 (d, J=12.38 Hz, 1H) 4.88-5.03 (m, 1H) 3.15 (br. s., 3H) 2.92 (br. s., 3H) 1.62 (d, J=6.82 Hz, 6H); MS (CI) m/z 275 (M⁺).

### Example 15(d)

### (2Z)-3-Dimethylamino-2-fluoro-1-(2-trifluoromethyl-1-isopropyl-1H-imidazol-5-yl)prop-2-en-1-one

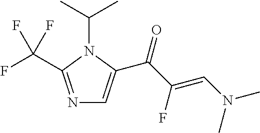

The title compound was prepared in accordance with the method described in Example 1(a), with the exception that the product did not need any purification and it was used crude in the next step. Using (2E)-3-dimethylamino-1-(2-trifluoromethyl-1-isopropyl-1H-imidazol-5-yl)prop-2-en-1-one (1.6 g, 5.8 mmol, obtained from Example 15(c)) the title compound was obtained (0.42 g, 25%). MS (CI) m/z 293 (M⁺).

### Example 15(e)

### 5-Fluoro-4-[2-trifluoromethyl-1-isopropyl-1H-imidazol-5-yl]pyrimidin-2-amine

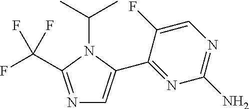

The title compound was prepared in accordance with the method described in Example 1(b) with the exception that guanidine carbonate was used. Using (2Z)-3-dimethylamino-2-fluoro-1-(2-trifluoromethyl-1-isopropyl-1H-imidazol-5-yl)prop-2-en-one (1.47 g, 5.22 mmol, obtained 15(d)) and guanidine carbonate (2.35 g, 13.06 mmol) the title compound (0.53 g, 33%) was obtained as a solid after purification by flash chromatography (CH₂Cl₂/MeOH 20:1).

¹H NMR (400 MHz, CHLOROFORM-d) δ ppm 8.28 (d, J=2.53 Hz, 1H) 7.53 (d, J=3.03 Hz, 1H) 5.04-5.14 (m, 1H) 5.02 (br. s., 2H) 1.61 (d, J=7.07 Hz, 6H); MS (ES) m/z 290 (M+1).

### Example 16

### [5-Fluoro-4-(3-isopropyl-2-trifluoromethyl-3H-imidazol-4-yl)-pyrimidin-2-yl]-[4-(4-methyl-piperazine-1-sulfonyl)-phenyl]-amine hydrochloride

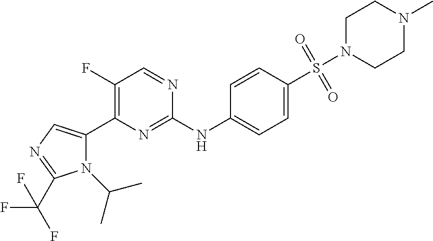

The title compound was prepared in accordance with the general method C using 5-fluoro-4-[2-trifluoromethyl-1-isopropyl-1H-imidazol-5-yl]pyrimidin-2-amine (obtained from Example 15(e)) (50 mg, 0.173 mmol), 1-[(4-bromophenyl)sulfonyl]-4-methylpiperazine (56 mg, 0.173 mmol), Cs₂CO₃ (114 mg, 0.350 mmol), Pd₂(dba)₃ (6 mg, 0.006 mmol) and X-Phos (5.3 mg, 0.011 mmol) to give the freebase of the title compound (10 mg, 3%) as a solid. The hydrochloride was prepared in accordance with the method described within general method B.

¹H NMR (400 MHz, CD₃OD) δ ppm 8.65 (s, 1H) 7.67-8.11 (m, 4H) 7.53 (s, 1H) 5.08-5.27 (m, 1H) 3.82-4.04 (m, 2H) 3.48-3.70 (m, 2H) 3.12-3.28 (m, 2H) 2.90 (s, 3H) 2.61-2.81 (m, 2H) 1.56 (d, J=5.30 Hz, 6H); MS (ES) m/z 528 (M+1).

### Example 17

### {4-[5-Fluoro-4-(3-isopropyl-2-trifluoromethyl-3H-imidazol-4-yl)-pyrimidin-2-ylamino]-phenyl}-(4-methyl-piperazin-1-yl)-methanone hydrochloride

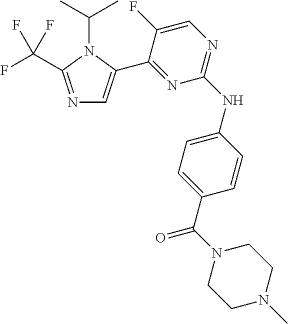

The title compound was prepared in accordance with the general method C using 5-fluoro-4-[2-trifluoromethyl-1-isopropyl-1H-imidazol-5-yl]pyrimidin-2-amine (obtained from Example 15(e)) (50 mg, 0.173 mmol), 1-(4-bromobenzoyl)-4-methylpiperazine (49 mg, 0.173 mmol), Cs₂CO₃ (114 mg, 0.350 mmol), Pd₂(dba)₃ (6 mg, 0.006 mmol) and X-Phos (5.3 mg, 0.011 mmol) to give the freebase of the title compound (25 mg, 29%) as a solid. The hydrochloride was prepared in accordance with the method described within general method B.

¹H NMR (400 MHz, CDCl₃) δ ppm 8.41-8.48 (m, 1H) 7.62 (d, J=8.59 Hz, 2H) 7.54 (d, J=3.03 Hz, 1H) 7.42 (d, J=8.59 Hz, 2H) 7.33 (s, 1H) 5.07-5.22 (m, 1H) 3.35-3.97 (m, 4H) 2.36-2.52 (m, 4H) 2.34 (s, 3H) 1.56 (d, J=7.07 Hz, 6H); MS (ES) m/z 490 (M−1).

### Example 18

### 3-{[5-Fluoro-4-(1-isopropyl-2-methyl-1H-imidazol-5-yl)pyrimidin-2-yl]amino}-N-(3-methoxypropyl)benzamide hydrochloride

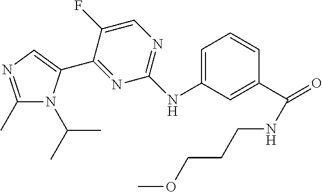

Methyl 3-{[5-fluoro-4-(1-isopropyl-2-methyl-1H-imidazol-5-yl)pyrimidin-2-yl]amino}benzoate (obtained from Example 18(a)) (40 mg, 0.11 mmol) and 3-methoxypropylamine (58 mg, 0.65 mmol) were mixed in toluene (4 mL) and an inert argon atmosphere was established. The sealed vial was cooled to r.t. and Al(CH₃)₃ (156 mg, 2.16 mmol) was added by a syringe. The reaction mixture was heated in an oil-bath at +90-100° C. for 4 h, cooled to r.t., and added dropwise into ice-cold sat. NaHCO₃ (aq) under vigorous stirring. The product was extracted with CH₂Cl₂ and the organic layer was dried by treatment with Na₂SO₄. The crude base of the product was purified using flash column chromatography (gradient from 100% EtOAc to 5% MeOH in EtOAc) and then the hydrochloride was prepared according to the method described within Example B to yield the title compound (8 mg, 16%).

¹H NMR (400 MHz, DMSO-d₆) δ ppm 9.90 (s, 1H) 8.78 (d, 1H) 8.39 (t, 1H) 8.07 (d, 1H) 8.01 (t, 1H) 7.78 (dd, 1H) 7.49-7.42 (m, 1H) 7.37 (t, 1H) 5.33-5.19 (m, 1H) 3.22 (s, 3H) 2.75 (s, 3H) 1.79-1.68 (m, 2H) 1.45 (s, 3H) 1.43 (s, 3H) some signals were partly obscured by the HDO signal; MS (ESI) m/z 427 (M+1).

### Example 18(a)

### Methyl 3-{[5-fluoro-4-(1-isopropyl-2-methyl-1H-imidazol-5-yl)pyrimidin-2-yl]amino}benzoate

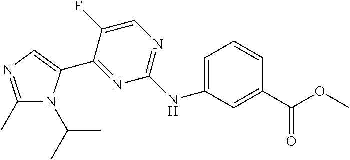

5-Fluoro-4-(1-isopropyl-2-methyl-1H-imidazol-5-yl)pyrimidin-2-amine (obtained from Example 1(b)) (99 mg, 0.42 mmol), methyl 3-bromobenzoate (97 mg, 0.45 mmol) and Cs₂CO₃ (230 mg, 0.71 mmol) were mixed in anhydrous 1,4-dioxane and the mixture was flushed with argon for 10 minutes before Pd₂(dba)₃ (23 mg, 0.025 mmol) and X-Phos (24 mg, 0.050 mmol) were added. The mixture was flushed with argon, then heated in a sealed tube at +90-100° C. until the reaction was complete. The reaction mixture was diluted with CH₂Cl₂, filtered and evaporated. The residue was taken up in CH₂Cl₂ and the organic phase was washed with H₂O. Residual water was removed from the organic phase by treatment with Na₂SO₄. The crude of the base product was purified using preparative HPLC to give the title compound (97 mg, 62%) as a solid.

¹H NMR (400 MHz, DMSO-d₆) δ ppm 9.73 (s, 1H) 8.57 (d, 1H) 8.19 (t, 1H) 8.04-7.95 (m, 1H) 7.59-7.52 (m, 1H) 7.43 (t, 1H) 7.37 (d, 1H) 5.44-5.31 (m, 1H) 3.84 (s, 3H) 2.51 (s, 3H) 1.42 (d, 3H) 1.40 (d, 3H); MS (ESI) m/z 370 (M+1).

### Pharmaceutical Compositions

According to one aspect of the present invention there is provided a pharmaceutical composition comprising a compound of formula I, as a free base or a pharmaceutically acceptable salt, solvate or solvate of salt thereof, for use in the prevention and/or treatment of conditions associated with glycogen synthase kinase-3.

The composition may be in a form suitable for oral administration, for example as a tablet, for parenteral injection as a sterile solution or suspension. In general the above compositions may be prepared in a conventional manner using pharmaceutically carriers or diluents. Suitable daily doses of the compounds of formula I in the treatment of a mammal, including man, are approximately 0.01 to 250 mg/kg bodyweight at peroral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

A compound of formula I, or a pharmaceutically acceptable salt, solvate or solvate of salt thereof, can be used on its own but will usually be administered in the form of a pharmaceutical composition in which the formula I compound/salt/solvate (active ingredient) is in association with a pharmaceutically acceptable excipient, diluent or carrier. Dependent on the mode of administration, the pharmaceutical composition may comprise from 0.05 to 99% w (percent by weight), for example from 0.10 to 50% w, of active ingredient, all percentages by weight being based on total composition.

An excipient, diluent or carrier includes water, aqueous polyethylene glycol, magnesium carbonate, magnesium stearate, talc, a sugar (such as lactose), pectin, dextrin, starch, tragacanth, microcrystalline cellulose, methyl cellulose, sodium carboxymethyl cellulose or cocoa butter.

A composition of the invention can be in tablet or injectable form. The tablet may additionally comprise a disintegrant and/or may be coated (for example with an enteric coating or coated with a coating agent such as hydroxypropyl methylcellulose).

The invention further provides a process for the preparation of a pharmaceutical composition of the invention which comprises mixing a compound of formula I, or a pharmaceutically acceptable salt, solvate or solvate of salt thereof, as hereinbefore defined, with a pharmaceutically acceptable excipient, diluent or carrier.

An example of a pharmaceutical composition of the invention is an injectable solution containing a compound of the invention, or a pharmaceutically acceptable salt, solvate or solvate of salt thereof, as hereinbefore defined, and sterile water, and, if necessary, either sodium hydroxide or hydrochloric acid to bring the pH of the final composition to about pH 5, and optionally a surfactant to aid dissolution.

### Medical Use

Surprisingly, it has been found that the compounds defined in the present invention, as a free base or a pharmaceutically acceptable salt thereof, are well suited for inhibiting glycogen synthase kinase-3 (GSK3). Accordingly, the compounds of the present invention are expected to be useful in the prevention and/or treatment of conditions associated with glycogen synthase kinase-3 activity, i.e. the compounds may be used to produce an inhibitory effect of GSK3 in mammals, including man, in need of such prevention and/or treatment.

GSK3 is highly expressed in the central and peripheral nervous system and in other tissues. Thus, it is expected that compounds of the invention are well suited for the prevention and/or treatment of conditions associated with glycogen synthase kinase-3 in the central and peripheral nervous system. In particular, the compounds of the invention are expected to be suitable for prevention and/or treatment of conditions associated with especially, dementia, Alzheimer's Disease, Parkinson's Disease, Frontotemporal dementia Parkinson's Type, Parkinson dementia complex of Guam, HIV dementia, diseases with associated neurofibrillar tangle pathologies and dementia pugilistica.

Other conditions are selected from the group consisting of amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss and contraceptive medication.

Further conditions are selected from the group consisting of predemented states, Mild Cognitive Impairment, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia

and Type I and Type II diabetes, diabetic neuropathy and diabetes related disorders.

One embodiment of the invention relates to the prevention and/or treatment of dementia and Alzheimer's Disease.

Another embodiment of the invention relates to the prevention and/or treatment of bone-related disorders.

The dose required for the therapeutic or preventive treatment of a particular disease will necessarily be varied depending on the host treated, the route of administration and the severity of the illness being treated.

The present invention relates also to the use of a compound of formula I as defined hereinbefore, in the manufacture of a medicament for the prevention and/or treatment of conditions associated with glycogen synthase kinase-3.

In the context of the present specification, the term “therapy” also includes “prevention” unless there are specific indications to the contrary. The terms “therapeutic” and “therapeutically” should be construed accordingly.

The invention also provides for a method of treatment and/or prevention of conditions associated with glycogen synthase kinase-3 comprising administering to a mammal, including man in need of such treatment and/or prevention a therapeutically effective amount of a compound of formula I, as hereinbefore defined.

### Non-Medical Use

In addition to their use in therapeutic medicine, the compounds of formula I as a free base or a pharmaceutically acceptable salt thereof, are also useful as pharmacological tools in the development and standardisation of in vitro and in vivo test systems for the evaluation of the effects of inhibitors of GSK3 related activity in laboratory animals such as cats, dogs, rabbits, monkeys, rats and mice, as part of the search for new therapeutics agents.

### Pharmacology

### Determination of ATP Competition in Scintillation Proximity GSK3β Assay.

### GSK3β Scintillation Proximity Assay.

The competition experiments were carried out in duplicate with 10 different concentrations of the inhibitors in clear-bottom microtiter plates (Wallac, Finland). A biotinylated peptide substrate, Biotin-Ala-Ala-Glu-Glu-Leu-Asp-Ser-Arg-Ala-Gly-Ser(PO₃H₂)-Pro-Gln-Leu (AstraZeneca, Lund), was added at a final concentration of 1 μM in an assay buffer containing 1 mU recombinant human GSK3β (Dundee University, UK), 12 mM morpholinepropanesulfonic acid (MOPS), pH 7.0, 0.3 mM EDTA, 0.01% β-mercaptorethanol, 0.004% Brij 35 (a natural detergent), 0.5% glycerol and 0.5 μg BSA/25 μl. The reaction was initiated by the addition of 0.04 μCi [γ-³³P]ATP (Amersham, UK) and unlabelled ATP at a final concentration of 1 μM and assay volume of 25 μl. After incubation for 20 minutes at room temperature, each reaction was terminated by the addition of 25 μl stop solution containing 5 mM EDTA, 50 μM ATP, 0.1% Triton X-100 and 0.25 mg streptavidin coated Scintillation Proximity Assay (SPA) beads (Amersham, UK). After 6 hours the radioactivity was determined in a liquid scintillation counter (1450 MicroBeta Trilux, Wallac). The inhibition curves were analysed by non-linear regression using GraphPad Prism, USA. The Kₘ value of ATP for GSK3β, used to calculate the inhibition constants (Kᵢ) of the various compounds, was 20 μM.

The following abbreviations have been used:

MOPS Morpholinepropanesulfonic acid
EDTA Ethylenediaminetetraacetic acid

### BSA Bovin Serum Albumin

### ATP Adenosine Triphosphate

### SPA Scintillation Proximity Assay

GSK3 Glycogen synthase kinase 3

### Results

Typical Kᵢ values for the compounds of the present invention are in the range of about 0.001 to about 10,000 nM. Other values for Kᵢ are in the range of about 0.001 to about 1000 nM. Further values for Kᵢ are in the range of about 0.001 nM to about 300 nM.

TABLE 1
Specimen results from assay.

| Example no | Kᵢ (nM) | n |
|---|---|---|
| 2 | 1.3 | 4 |
| 5 | 24 | 4 |
| 11 | 25 | 3 |
| 13 | 3.8 | 3 |

1. Use of a compound of formula I, 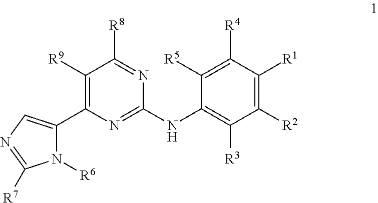
wherein
R¹ is selected from hydrogen, halo, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};
R² and R⁴ are independently selected from hydrogen, halo, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};
R³ and R⁵ independently are selected from hydrogen, C₁₋₃alkyl, C₁₋₃haloalkyl and OR^{a};
R⁶ is selected from C₂₋₄alkyl, C₂₋₄alkenyl, C₂₋₄alkynyl, and C₂₋₄haloalkyl;
R⁷ is selected from C₁₋₃alkyl, CN, and C₁₋₃haloalkyl, said C₁₋₃alkyl or C₁₋₃haloalkyl optionally substituted with one or more OR^{a};
R⁸ and R⁹ are independently selected from hydrogen, CN and halo;
R^{a} is hydrogen, C₁₋₃alkyl or C₁₋₃haloalkyl, said C₁₋₃alkyl or C₁₋₃haloalkyl optionally substituted with one or more C₁₋₃alkoxy;
R^{b} and R^{c} are independently selected from hydrogen, C₁₋₆alkyl or C₁₋₆haloalkyl, said C₁₋₆alkyl or C₁₋₆haloalkyl optionally substituted with one or more OR^{a} or NR^{d}R^{e}; or
R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;
R^{d} and R^{e} are independently selected from hydrogen, C₁₋₆alkyl or C₁₋₆haloalkyl, said C₁₋₆alkyl or C₁₋₆haloalkyl optionally substituted with one or more OR^{a}; or
R^{d} and R^{e} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;
R^{h} is hydrogen, C₁₋₃alkyl or C₁₋₃haloalkyl, said C₁₋₃alkyl or C₁₋₃haloalkyl optionally substituted with one or more C₁₋₃alkoxy;
Rⁱ is C₁₋₃alkyl or C₁₋₃haloalkyl, said C₁₋₃alkyl or C₁₋₃haloalkyl optionally substituted with one or more OR^{a}; and
is R^{j} is an aryl or heteroaryl ring, wherein said aryl or heteroaryl ring is optionally substituted with one or more C₁₋₃alkyl, OR^{a}, halo or CN;
or a pharmaceutically acceptable salt thereof for use in the manufacturing of a medicament for prevention and/or treatment of Alzheimer's Disease.

2. The use of according to claim 1, wherein
R¹ is selected from hydrogen, SO₂NR^{b}R^{c}, C(O)NR^{b}R^{c}, CH₂NR^{b}R^{c} and C(O)R^{j};
R² and R⁴ are independently selected from hydrogen, halo, CN, C₁₋₃alkyl, OR^{a}, and SO₂Rⁱ;
R³ and R⁵ independently are selected from hydrogen, C₁₋₃alkyl, C₁₋₃haloalkyl;
R⁶ is selected from C₂₋₄alkyl, C₂₋₄alkenyl, C₂₋₄alkynyl, and C₂₋₄haloalkyl;
R⁷ is C₁₋₃alkyl;
R⁸ and R⁹ are independently selected from hydrogen and halo; and
R^{a} is C₁₋₃alkyl or C₁₋₃haloalkyl, said C₁₋₃alkyl or C₁₋₃haloalkyl optionally substituted with one or more C₁₋₃alkoxy;
R^{b} and R^{c} are independently selected from hydrogen, C₁₋₆alkyl or C₁₋₆haloalkyl, said C₁₋₆alkyl or C₁₋₆haloalkyl optionally substituted with one or more OR^{a}; or
R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;
Rⁱ is C₁₋₃alkyl; and
R^{j} is aryl or heteroaryl;
or a pharmaceutically acceptable salt thereof.

3. The use according to claim 1 or 2, wherein
R¹ is selected from SO₂NR^{b}R^{c}, C(O)NR^{b}R^{c} and C(O)R^{j};
R² and R⁴ are independently selected from hydrogen, halo, CN, C₁₋₃alkyl, OR^{a}, and SO₂Rⁱ;
R³ and R⁵ independently are selected from hydrogen, C₁₋₃alkyl, C₁₋₃haloalkyl;
R⁶ is C₂₋₄alkyl;
R⁷ is C₁₋₃alkyl;
R⁸ and R⁹ are independently selected from hydrogen and halo;
R^{a} is C₁₋₃alkyl or C₁₋₃haloalkyl;
R^{b} and R^{c} may, together with the atom to which they are attached, form a 4-, 5 or 6-membered heterocyclic ring containing one or more heteroatoms selected from N, O or S, wherein said heterocyclic ring is optionally substituted with one or more C₁₋₃alkyl;
Rⁱ is C₁₋₃alkyl; and
R^{j} is aryl or heteroaryl;
or a pharmaceutically acceptable salt thereof.

4. The use according to claim 1, wherein R⁹ is halo and R⁸ is hydrogen.

5. The use according to claim 4, wherein R⁹ is fluoro.

6. The use according to claim 4 or claim 5, wherein R⁶ is C₂₋₄alkyl.

7. The use according to claim 6, wherein R⁶ is isopropyl.

8. The use according to any one of claims 4 to 7, wherein R⁷ is fluoromethyl or methyl.

9. The use according to any one of claims 4 to 8, wherein R² and R⁴ are hydrogen.

10. The use according to any one of claims 4 to 9, wherein R⁵ and R³ are hydrogen.

11. The use according to any one of claims 4 to 10, wherein R¹ is selected from C(O)NR^{b}R^{c}, SO₂R^{b}R^{c}, SO₂Rⁱ or C(O)R^{j}.

12. The use according to claim 11, wherein R^{j} is phenyl or piperidin.

13. The use according to claim 11, wherein R^{b} and R^{c}, together with the atom to which they are attached, form a 6-membered heterocyclic ring containing one or more heteroatoms selected from N, wherein said heterocyclic ring is optionally substituted with one or more halo, C₁₋₃alkyl or C₁₋₃haloalkyl.

14. The use according to claim 13, wherein said heterocyclic ring is substituted with one or more C₁₋₃alkyl.

15. The use according to claim 14, wherein said C₁₋₃alkyl is methyl.

16. The use according to claim 11, wherein Rⁱ is C₁₋₃alkyl.

17. The use according to claim 11, wherein Rⁱ is methyl,

18. The use according to claim 1, said compound being selected from:
(4-{[5-Fluoro-4-(1-isopropyl-2-methyl-1H-imidazol-5-yl)pyrimidin-2-yl]amino}phenyl)(phenyl)methanone hydrochloride;
(4-{[5-Fluoro-4-(1-isopropyl-2-methyl-1H-imidazol-5-yl)pyrimidin-2-yl]amino}phenyl)(pyridin-2-yl)methanone hydrochloride;
(4-{[5-Fluoro-4-(1-isopropyl-2-methyl-1H-imidazol-5-yl)pyrimidin-2-yl]amino}phenyl)(pyridin-3-yl)methanone hydrochloride;
5-Fluoro-4-(1-isopropyl-2-methyl-1H-imidazol-5-yl)-N-{3-methyl-4-[(4-methylpiperazin-1-yl)sulfonyl]phenyl}pyrimidin-2-amine hydrochloride;
5-Fluoro-4-(1-isopropyl-2-methyl-1H-imidazol-5-yl)-N-{2-methyl-4-[(4-methylpiperazin-1-yl)sulfonyl]phenyl}pyrimidin-2-amine hydrochloride;
5-Fluoro-4-(1-isopropyl-2-methyl-1H-imidazol-5-yl)-N-[4-[(4-methylpiperazin-1-yl)sulfonyl]-2-(trifluoromethyl)phenyl]pyrimidin-2-amine hydrochloride;
5-Fluoro-4-(1-isopropyl-2-methyl-1H-imidazol-5-yl)-N-[4-[(4-methylpiperazin-1-yl)sulfonyl]-3-(trifluoromethoxy)phenyl]pyrimidin-2-amine hydrochloride;
5-Fluoro-4-(1-isopropyl-2-methyl-1H-imidazol-5-yl)-N-[4-[(4-methylpiperazin-1-yl)carbonyl]-3-(methylsulfonyl)phenyl]pyrimidin-2-amine hydrochloride;
5-{[5-Fluoro-4-(1-isopropyl-2-methyl-1H-imidazol-5-yl)pyrimidin-2-yl]amino}-2-[(4-methylpiperazin-1-yl)carbonyl]benzonitrile hydrochloride;
N-{3-Chloro-4-[(4-methylpiperazin-1-yl)carbonyl]phenyl}-5-fluoro-4-(1-isopropyl-2-methyl-1H-imidazol-5-yl)pyrimidin-2-amine hydrochloride;
5-Fluoro-4-(1-isopropyl-2-methyl-1H-imidazol-5-yl)-N-{3-methoxy-4-[(4-methylpiperazin-1-yl)carbonyl]phenyl}pyrimidin-2-amine hydrochloride;
(4-{[5-Fluoro-4-(1-isopropyl-2-methyl-1H-imidazol-5-yl)pyrimidin-2-yl]amino}phenyl)(pyridin-4-yl)methanone hydrochloride;
5-Fluoro-4-(1-isopropyl-2-methyl-1H-imidazol-5-yl)-N-{4-[(4-methylpiperazin-1-yl)carbonyl]phenyl}pyrimidin-2-amine hydrochloride;
5-Fluoro-4-(1-isopropyl-2-methyl-1H-imidazol-5-yl)-N-{4-[(4-methylpiperazin-1-yl)sulfonyl]phenyl}pyrimidin-2-amine hydrochloride;
5-Fluoro-4-[1-isopropyl-2-(trifluoromethyl)-1H-imidazol-5-yl]-N-[4-(methylsulfonyl)phenyl]pyrimidin-2-amine hydrochloride;
5-Fluoro-4-[1-isopropyl-2-(trifluoromethyl)-1H-imidazol-5-yl]-N-[4-(methylsulfonyl)phenyl]pyrimidin-2-amine hydrochloride;
{4-[5-Fluoro-4-(3-isopropyl-2-trifluoromethyl-3H-imidazol-4-yl)-pyrimidin-2-ylamino]-phenyl}-(4-methyl-piperazin-1-yl)-methanone hydrochloride; and
3-{[5-Fluoro-4-(1-isopropyl-2-methyl-1H-imidazol-5-yl)pyrimidin-2-yl]amino}-N-(3-methoxypropyl)benzamide hydrochloride;
or a pharmaceutically acceptable salt thereof.

19. A pharmaceutical formulation for use in the treatment and/or prophylaxis of Alzheimer's Disease, comprising a therapeutically effective amount of a compound of formula I as defined in any one of claims 1 to 18 or a pharmaceutically acceptable salt thereof and conventional excipients.

20. A method of treatment and/or prophylaxis of Alzheimer's Disease comprising administering to a mammal, including man in need of such treatment and/or prophylaxis a therapeutically effective amount of a compound of formula I as defined in any one of claims 1 to 18 or a pharmaceutically acceptable salt thereof.

21. A compound selected from:
1-[4-Bromo-2-(methylsulfonyl)benzoyl]-4-methylpiperazine;
1-(4-Chloro-2-iodobenzoyl)-4-methylpiperazine;
5-Chloro-2-[(4-methylpiperazin-1-yl)carbonyl]benzonitrile;
1-(4-Chloro-2-methoxybenzoyl)-4-methylpiperazine;
2,2,2-Trifluoro-N-isopropyl-N-(5-methyl-isoxazol-4-yl)-acetamide;
5-Acetyl-2-trifluoromethyl-1-isopropyl-1H-imidazole;
(2E)-3-Dimethylamino-1-(2-trifluoromethyl-1-isopropyl-1H-imidazol-5-yl)prop-2-en-1-one;
(2Z)-3-Dimethylamino-2-fluoro-1-(2-trifluoromethyl-1-isopropyl-1H-imidazol-5-yl)prop-2-en-1-one; and
5-Fluoro-4-[2-trifluoromethyl-1-isopropyl-1H-imidazol-5-yl]pyrimidin-2-amine;
Methyl 3-{[5-fluoro-4-(1-isopropyl-2-methyl-1H-imidazol-5-yl)pyrimidin-2-yl]amino}benzoate.

22. Use of the compounds according to claim 21 in the preparation of a compound of formula I as defined in claim 1.
